(19) United States
(12) Patent Application Publication
(10) Pub. No.: US 2023/0348674 A1
(43) Pub. Date: Nov. 2, 2023

(54) HYDROGELS

(71) Applicant: Commonwealth Scientific and Industrial Research Organisation, Acton (AU)

(72) Inventors: Anh Tuan Nguyen, Victoria (AU); Johan Basuki, Victoria (AU); Timothy Hughes, Victoria (AU)

(21) Appl. No.: 18/012,815

(22) PCT Filed: Jun. 24, 2021
(86) PCT No.: PCT/AU2021/050659

§ 371 Date: Dec. 23, 2022

(30) Foreign Application Priority Data
Jun. 24, 2020 (AU) 2020902116

(51) Int. Cl. C08J 3/075; A61K 9/06; A61K 38/38; A61K 47/36; A61K 47/34; C08G 73/02

(52) U.S. Cl. CPC C08J 3/075; A61K 9/06; A61K 38/385; A61K 47/34; A61K 47/36; C08G 73/0233; C08G 2210/00; C08G 2230/00; C08J 2379/02; C08J 2389/06

(57) ABSTRACT

Described herein is a method of preparing a thermoresponsive polymer biomacromolecule conjugate, the method comprising: preparing by living cationic ring opening polymerisation a thermoresponsive polymer selected from polyoxazoline, polyoxazine, or copolymer thereof, the so-formed thermoresponsive polymer having a living cation; and reacting the living cation with a nucleophilic functional group selected from carboxylate, amino, sulfate, sulfonate, phosphate, phosphonate and thiol of a biomacromolecule to conjugate the thermoresponsive polymer to the biomacromolecule; wherein in an aqueous liquid the so-formed thermoresponsive polymer biomacromolecule conjugate exhibits a gelation temperature and forms a hydrogel in that liquid above that gelation temperature.

### FIELD OF THE INVENTION

The present invention relates in general to hydrogels. The invention relates more particularly to a method of preparing biomacromolecule thermoresponsive polymer conjugates that exhibit in an aqueous liquid a gelation temperature and form a hydrogel in the aqueous liquid above that gelation temperature.

### BACKGROUND OF THE INVENTION

Hydrogels have found application in many biomedical applications owing to their high water content, mechanical properties and good biocompatibility. A particularly useful application of hydrogels is where they are formulated to comprise a biologically active agent, for example, a drug, which can diffuse out from the hydrogel in a controlled manner when in implanted in a subject. Such hydrogels can be designed to degrade in situ after release of the biologically active agent to assist with clearance from the subject.

In certain applications it may be desirable for a hydrogel to present initially in a free-flowing liquid state so as to be injectable then, upon being injected at a desired location in a subject, transition into a gel state. For example, the hydrogel may present in a free-flowing liquid state at room temperature and upon being injected into a subject transition into a gel state at body temperature. Hydrogels with such properties are known and are commonly referred to in the art as thermoresponsive or thermoreversible hydrogels.

Known thermoresponsive hydrogels include those based on a biomacromolecule (such as polysaccharide) conjugated with thermoresponsive polymer (such as poly (N-isopropylacrylamide) (pNIPAM)).

While such thermoresponsive hydrogels are known, the current methodology for producing them is typically complex and inherently limited in scope. For example, conventional reaction protocol for producing a biomacromolecule thermoresponsive polymer conjugate typically involves multiple reaction steps. Also, such conventional reaction protocols are prone to producing conjugates that simply fail to exhibit desired thermoresponsive hydrogel behavior (e.g., the so formed conjugate is prone to precipitation rather than hydrogel formation, or if a hydrogel can form it exhibits a low storage modulus (G′) and therefore presents poor gel properties).

An opportunity therefore remains to develop methodology for preparing biomacromolecule thermoresponsive polymer conjugates in a simple efficient manner that provides an ability to control and tailor the thermoresponsive and gel characteristics of hydrogels formed using the conjugates.

### SUMMARY OF THE INVENTION

The present invention provides a method of preparing a thermoresponsive polymer biomacromolecule conjugate, the method comprising
preparing by living cationic ring opening polymerisation a thermoresponsive polymer selected from polyoxazoline, polyoxazine and copolymers thereof, the so formed thermoresponsive polymer having a living cation; and
reacting the living cation with a nucleophilic functional group selected from carboxylate, amino, sulfate, sulfonate, phosphate, phosphonate and thiol of a biomacromolecule to conjugate the thermoresponsive polymer to the biomacromolecule;
wherein in an aqueous liquid the so formed thermoresponsive polymer biomacromolecule conjugate exhibits a gelation temperature and forms a hydrogel in the aqueous liquid above that gelation temperature.

The method of the invention has surprisingly been found to provide an ability to prepare thermoresponsive polymer biomacromolecule conjugates in an effective and efficient manner and also provide an ability to control and tailor the thermoresponsive and gel characteristics of hydrogels formed using the conjugates.

According to the method, the specified thermoresponsive polymer is prepared by living ring opening cationic polymerisation. The so-formed thermoresponsive polymer presents a living cation which then reacts with the specified nucleophilic functional groups on the biomacromolecule. That reaction conjugates the thermoresponsive polymer to the biomacromolecule. Both the polymerisation and conjugation reactions can advantageously take place in a so-called “one-pot” procedure.

Use of a biomacromolecule with the specified nucleophilic functional groups surprisingly enables conjugation of the thermoresponsive polymer to the biomacromolecule to take place in a controlled and tailored manner. Such reaction control advantageously enables control over the degree of substitution of thermoresponsive polymer conjugated to the biomacromolecule, which in turn has surprisingly been found to significantly influence the thermoresponsive and gel characteristics of hydrogels formed using the conjugates.

Without wishing to be limited by theory, it is believed the unique combination of the specific thermoresponsive polymer conjugated to a biomacromolecule (i) through a cationic reaction mechanism, and (ii) via specific nucleophilic functional groups, affords excellent conjugation control and advantageously enables the thermoresponsive and gel properties of hydrogels formed using the conjugates to be tailored as required.

In one embodiment, the living cationic ring opening polymerisation prepares a poly-2- oxazoline, poly-2-oxazine, or copolymer thereof. In a further embodiment, the poly-2-oxazoline is a poly(2-alkyl-2-oxazoline) and the poly-2-oxazine is a poly(2-alkyl-2-oxazine).

In a further embodiment, the biomacromolecule is selected from a polysaccharide and polypeptide.

In one embodiment, the polysaccharide is hyaluronic acid (HA).

In another embodiment, the polypeptide is gelatin.

In a further embodiment, the hydrogel in the aqueous liquid has a storage modulus (G′) of greater than 100 Pa at >37° C., as measured by a rheometer.

The present invention also provides a method of forming a hydrogel, the method comprising: providing an aqueous liquid solution of thermoresponsive polymer biomacromolecule conjugate prepared in accordance with the method of the invention, the thermoresponsive polymer biomacromolecule conjugate exhibiting in the aqueous liquid a gelation temperature; and

raising the temperature of the aqueous liquid comprising the thermoresponsive polymer biomacromolecule conjugate above the gelation temperature so as to promote formation of the hydrogel.

Further aspects and/or embodiments of the invention are discussed in more detail below.

### BRIEF DESCRIPTION OF THE DRAWINGS

The present invention will herein be described with reference to the following nonlimiting drawings in which:

FIG. 1 illustrates the storage modulus (G′) and loss modulus (G″) of Conjugate 6 as a function of concentration (in % w/v) in aqueous phosphate buffer solution (PBS), measured by rheometer at 37° C. Hydrogel formation is indicated when G′ is higher than G″,

FIG. 2 illustrates the tangent δ (or G″/G′ ratio) measured by rheometer at 37° C. of Conjugate 6 as a function of concentration (in % w/v) in aqueous phosphate buffer solution (PBS). Hydrogel formation is indicated when tangent δ is less than 1;

FIG. 3 illustrates the relationship between rheological properties (G′ and G″) and concentration of the conjugates (Conjugates 2, 7, 11) synthesised by using different HA’s molecular weight and POxa’s LCST. Conjugates 2 and 7 with similar DS = 16% were prepared from HA with 100 kDa and 500 kDa molecular weight, respectively. While the HA-POxa conjugates were prepared with similar DS = 16% and 500 kDa HA, Conjugates 7 and 11 contain POxa with different LCST (24° C. and 17° C., respectively);

FIG. 4 illustrates the relationship between degree of substitution (DS) and rheological moduli (G′ and G″) measured at 37° C. of HA-POxa conjugates (Conjugates 3 10) at different weight concentrations (7% and 18% w/v) in aqueous phosphate buffer solution (PBS). The optimal DS range of the HA-POxa conjugate to form a hydrogel was determined, when the G′ is higher than G″ 37° C.;

FIG. 5 illustrates HRP release kinetics profiles over 7 days of the hydrogels from Conjugate 7 and Conjugate 11 at 18% w/v concentration in aqueous phosphate buffer solution (PBS). For comparison, POxa and POxa/HA Mixture (a physical mixture of HA and POxa) were used as negative controls;

FIG. 6 illustrates biological activity of the HRP released from the hydrogels from Conjugate 7 and Conjugate 11 (18% w/v) after incubated over 7 days at 37° C. For comparison, POxa and POxa/HA Mixture (a physical mixture of HA and POxa) were used as negative controls. Fresh HRP solution was used as a standard sample to depict 100% bioactivity, whereby the HRP solution after 7-days incubation at 37° C. (PBS 1x) serves as negative control as well;

FIG. 7 illustrates the relationship between degree of substitution (DS) and rheological moduli (G′ and G″) measured at 37° C. of Gelatin-POxa conjugates (Conjugates 12 - 18) at 20% w/v in aqueous phosphate buffer solution (PBS). Prior to ‘one-pot’ conjugation, the porcine gelatin (bloom 300) chemically pre-modified using ethylenediamine (pre-aminated gelatin) to provide amino functional groups for ‘one-pot’ POxa conjugation. The optimal DS range of the Gelatin-POxa conjugate to form a hydrogel was determined, when the G′ is higher than G″ 37° C.;

FIG. 8 illustrates rheological properties (G′ and G″) of a physical mixture of 7% w/v HA-POXa Conjugate 6 and 5% w/v pre-aminated gelatin (Mixture 1) in comparison with 7% w/v of Conjugate 6 only in aqueous phosphate buffer solution (PBS); and

FIG. 9 illustrates Rheological properties of (G′ and G″) of a physical mixture of 500 kDa HA and 13.6 kDa POxa (HA/POxa Mixture) in comparison with Conjugate 7 at the same 7% w/v concentration in aqueous phosphate buffer solution (PBS).

FIG. 10 illustrates BSA release kinetics profile over two weeks from the hydrogel, Conjugate 12, BSA at 10% w/v concentration in aqueous phosphate buffer solution (PBS 1x).

FIG. 11 illustrates release kinetics profile of Ephrin-A1 construct over three weeks from the hydrogel, Conjugate 12 - Ephrin-A1 at 7% w/v concentration in aqueous phosphate buffer solution (PBS 1x).

FIG. 12 illustrates release kinetics profile of Sodium Fluorescein over six weeks from the hydrogel, Conjugate 6 - Sodium Fluorescein T18% w/v concentration in aqueous phosphate buffer solution (PBS 1x).

### DETAILED DESCRIPTION OF THE INVENTION

The present invention provides a method of preparing a thermoresponsive polymer biomacromolecule conjugate.

Reference herein to “thermoresponsive polymer” it is intended to mean polymer that exhibits in an aqueous liquid a Lower Critical Solution Temperature (LCST). Those skilled in the art will appreciate that upon being subjected to a change in temperature thermoresponsive polymers can undergo a conformational molecular rearrangement and transition from being hydrophilic in character (i.e. soluble in the aqueous liquid) to being hydrophobic in character (i.e. insoluble in the aqueous liquid), with that transition being reversible. As that transition promotes a change in solubility of the polymer in the aqueous liquid it can typically be characterized using UV-Visible transmission spectroscopy by observing the change in aqueous solution turbidity as a function of temperature. When cloud point temperatures are plotted against the concentration of the polymer, the minimum cloud point temperature is defined as the LCST of the polymer. Further procedural details for determining the LCST of a polymer is provided in the Example section below.

In the context of the present invention the LCST property of the thermoresponsive polymer advantageously carries over to the thermoresponsive polymer biomacromolecule conjugate itself whereby in an aqueous liquid the conjugate can be seen to also exhibit a LCST. While the LCST of the conjugate is derived from the thermoresponsive polymer, due to the polymer being conjugated to the biomacromolecule it will generally be different to that of the thermoresponsive polymer per se. The LCST of the conjugate can be determined in the same way as the LCST of the thermoresponsive polymer outlined above.

The LCST of the conjugate represents a transition point that can lead to hydrogel formation, as is required in accordance with the invention. In particular, when passing through the conjugate LCST to a temperature above that LCST the thermoresponsive polymer still undergoes a conformational molecular rearrangement and transitions from being soluble in the aqueous liquid to being insoluble in the aqueous liquid. That conformational molecular rearrangement correspondingly reduces solubility of the conjugate in the aqueous liquid and can give rise to hydrogel formation through molecular interactions between separate conjugates driven by entanglement of the thermoresponsive polymer substituents.

The temperature at which the conjugate begins transitioning into a hydrogel in the aqueous liquid is defined herein as its “gelation temperature”. The gelation temperature of the conjugate can be conveniently measured using a rheometer and is identified at a temperature when the storage modulus (G′) and the loss modulus (G″) are equal (i.e. tan δ = 1). Further procedural details for determining the gelation temperature of conjugate is provided in the Example section below.

While the LCST and the gelation temperature of the conjugate may be the same, that may not always be the case. Where the LCST and the gelation temperature of the conjugate are different, the gelation temperature will typically be above (for example, up to about 5° C., 10° C., 15° C. or 20° C. above) the LCST.

As will be discussed in more detail below, just because a conventional thermoresponsive polymer biomacromolecule conjugate exhibits an LCST that does not necessarily mean it will also exhibit a gelation temperature. The present application advantageously provides teaching to produce in an effective and efficient manner thermoresponsive polymer biomacromolecule conjugates that exhibit both an LCST and gelation temperature.

The thermoresponsive polymer used in accordance with the invention is selected from polyoxazoline, polyoxazine and copolymers thereof.

In one embodiment, the thermoresponsive polymer is selected from poly(2-oxazoline), poly(2-oxazine) and copolymers thereof.

In another embodiment, the thermoresponsive polymer is selected from poly(2-alkyl-2-oxazoline), poly(2-alkyl-2-oxazine) and copolymers thereof. In one embodiment, the alkyl group is C₁-C₁₂ alkyl, or C₁-C₈ alkyl, or C₁-C₆ alkyl.

As will be discussed in more detail below, the thermoresponsive polymer is prepared by living cationic ring opening polymerisation of appropriate monomer.

The thermoresponsive properties of thermoresponsive polymer used in accordance with the invention, and consequently the thermoresponsive properties of the so-formed conjugate, can be adjusted through parameters such as the molecular weight of the thermoresponsive polymer and the composition of polymerised monomer residues that make up the thermoresponsive polymer, including the location, type and concentration of polymerized monomer residues in the polymer chain. For example, the LCST of a given thermoresponsive polymer can be adjusted through variation in the composition of polymerised monomer residues that make up the polymer chain.

Those skilled in the art will be familiar with tailoring the LCST of a thermoresponsive polymer through variation of the monomer type and concentration used to make the polymer. Tailoring of the LCST of a given thermoresponsive polymer can correspondingly influence the LCST and gelation temperature of the conjugate. For example, reducing the LCST of the polymer can reduce both the LCST and gelation temperature of the conjugate.

An important feature of the present invention is use of a biomacromolecule. As used herein, the term “biomacromolecule” is intended to mean a molecule with a molecular mass exceeding 1 kDa derived from live organisms or a polymer of biological origin comprising sequential monomeric units and having nucleophilic functional groups, which are available inherently or introduced synthetically through a chemical modification. A biomacromolecule according to the present invention will be constructed from monomeric units joined together in a repeating sequence. Examples of such monomeric units include saccharide (e.g. uronic acid, amino sugar, etc.) and amino acid (e.g. lysine, aspartate, glutamate, cysteine etc.). Those monomeric units may be joined together in a repeating sequence to form, for example, biomacromolecules selected form polysaccharides, and polypeptides.

Biomacromolecules used in accordance with the invention are ones that incorporate as part of their molecular structure nucleophilic functional groups selected from carboxylate (—COO⁻), amino (primary, secondary, or tertiary), sulphate (—OS(O)(O)O⁻), sulfonate (—S(O)(O)O⁻), phosphate (—OP(O)O, phosphonate (—P(O)O⁻O⁻) and thiol (—SH). The biomacromolecule can have one or a combination of the specified nucleophilic functional groups. The biomacromolecule will typically have a plurality of such nucleophilic functional groups.

Those skilled in the art will appreciate that biomacromolecules that do not natively contain one of the aforementioned nucleophilic functional groups can be modified through chemical reaction to derivatise a functional group into one of the specified nucleophilic functional groups. Accordingly, biomacromolecules suitable for use in accordance with the invention include biomacromolecules that have been derivatised so as to present one or more of the nucleophilic functional groups specified for use in accordance with the invention. The biomacromolecules may also be chemically modified so as to convert one or more of the nucleophilic functional groups specified for use in accordance with the invention into a different nucleophilic functional group(s) specified for use in accordance with the invention (e.g. carboxylate to amino).

For avoidance of any doubt, it is to be assumed that reference herein to a biomacromolecule being used in accordance with the invention is a reference to a biomacromolecule comprising one or more nucleophilic functional groups selected from carboxylate, amino, sulphate, sulfonate, phosphate, phosphonate and thiol.

In one embodiment, the biomacromolecule is selected from a polysaccharide and a polypeptide.

Those skilled in the art will appreciate a polypeptide is in effect a polymeric form of amino acids. Typical amino acid units that form part of polypeptide suitable for use in accordance with the invention include, but are not limited to, aspartate, glutamate, lysine, and cysteine. There is no particular limitation on the molecular weight of the polypeptide that may be used in accordance with the invention. However, the molecular weight of such polypeptides will generally range from about 5 kDa to about 500 kDa.

Suitable examples of polypeptides that may be used in accordance with the invention include, but are not limited to, gelatin and collagen.

When used, the collagen may have a molecular weight of from about 100 kDa to about 300 kDa.

In one embodiment, the collagen is selected from Type I, type II and type IV collagen.

When used, the gelatin may a molecular weight of from about 10 kDa to about 100 kDa.

Sources of gelatin can include the extraction and hydrolysis of collagen from biological tissue (e.g. porcine skin) or by biotechnological fermentation (e.g. Escherichia coli).

In one embodiment, the gelatin used is chemically modified gelatin. For example, without particular limitation, native carboxylate groups of gelatin from aspartate and glutamate can be chemically modified (e.g. with alkyldiamine) to provide aminated gelatin.

Those skilled in the art will appreciate that polysaccharides are complex carbohydrates made up of monomeric saccharide units joined together by glycosidic bonds. Typical saccharide units that form part of polysaccharides suitable for use in accordance with the invention include, but are not limited to a uronic acid, for example glucuronic acid, iduronic acid, mannuronic acid, and galacturonic acid, and an amino sugar, for example glucosamine, glucosamine, and galactosamine. The saccharide units can be inherently sulphated or acetylated in their native forms.

Polysaccharides containing those saccharide units include, but are not limited to, hyaluronic acid (hyaluronan), mannuronan, heparin, heparan sulphate, chondroitin sulphate, dermatan sulphate, keratan sulphate, ulvan, chitosan, alginic acid, gellan gum, xanthan gum, and pectin.

There is no particular limitation on the molecular weight of polysaccharides that may be used in accordance with the invention. However, the molecular weight will typically range from about 5 kDa to about 10,000 kDa.

Typical polysaccharides suitable for use in accordance with the invention include, but are not limited to, glycosaminoglycan and other biological polysaccharides that comprise uronic acid and/or amino sugar units, for example glucuronic acid or iduronic acid and glucosamine, respectively.

In one embodiment, the biomacromolecule is hyaluronic acid (HA).

The HA may be provided in the form of a salt thereof, for example as a sodium, potassium, phosphonium or ammonium salt thereof.

In one embodiment, HA is in the form of a tetra-alkyl (e.g. C₂-C₆) or tetra-aryl phosphonium or ammonium salt.

In another embodiment, HA is in the form of a tetrabutyl ammonium salt.

The HA may have a molecular weight of from about 20 kDa to about 10,000 kDa, for example about 50 kDa to about 2,500 kDa, or from about 100 kDa to about 1,000 kDa.

Sources of HA include extraction from biological tissue (e.g. bovine vitrous humor) or by biotechnological fermentation (e.g. Streptococcus zooepidemicus).

In one embodiment, the HA used is chemically modified HA. For example, without particular limitation, native carboxylate of HA can be chemically modified (e.g. with alkyldiamine) to provide aminated HA. N-acetyl glucosamine unit of HA can also be deacetylated to provide aminated HA.

The product produced in accordance with the method of the invention is a thermoresponsive polymer biomacromolecule conjugate. By being a “conjugate” is meant the thermoresponsive polymer is covalently coupled (i.e., conjugated) to the biomacromolecule. As will be appreciated from the method of the invention and as will be discussed in more detail below, that covalent coupling or conjugation occurs through the specified nucleophilic functional group that form part of the biomacromolecule.

For convenience, the thermoresponsive polymer biomacromolecule conjugate prepared in accordance with the present invention may simply be referred to herein as the “conjugate”.

To afford the conjugate, the method according to the invention comprises preparing by living cationic ring opening polymerisation a thermoresponsive polymer selected from polyoxazoline, polyoxazine and copolymers thereof.

Preparation of polyoxazoline, polyoxazine or copolymers thereof, by living cationic ring opening polymerisation is known in the art. Known techniques, equipment, and reagents for producing those polymers can advantageously be used in accordance with the invention.

In one embodiment, the method comprises preparing by living cationic ring opening polymerisation a thermoresponsive polymer selected from poly(2-oxazoline), poly(2-oxazine) and copolymers thereof.

In another embodiment, the poly(2-oxazoline) is a poly(2-alkyl-2-oxazoline).

In a further embodiment, the poly(2-oxazine) is a poly(2-alkyl-2-oxazine).

The alkyl group in the oxazoline and oxazine (co)polymers may be C₁-C₁₂ alkyl, or C₁-C₈ alkyl or C₁-C₆ alkyl.

In one embodiment, the method comprises preparing by living cationic ring opening polymerisation a thermoresponsive polymer selected from poly(2-ethyl-2-oxazoline), poly(2-isopropyl-2-oxazoline), poly(2-n-propyl-2-oxazoline), poly(2-n-butyl-2-oxazoline), poly(2-N,N-diethylamino-2-oxazoline), poly(n-propyl-2-oxazine) and copolymers thereof.

In a further embodiment, the thermoresponsive polymer prepared by the living cationic ring opening polymerisation has a molecular weight of between about 1 kDa and about 150 kDa, or about 5 kDa and about 100 kDa, or about 10 kDa and about 40 kDa.

Reference herein to a molecular weight of a thermoresponsive polymer is a number average molecular weight (Mn) measured by gel permeation chromatography (GPC) as outlined in the Example section below.

Those skilled in the art will appreciate the polyoxazoline, polyoxazine, or copolymers thereof prepared in accordance with the invention will be formed through polymerisation of suitable oxazoline and/or oxazine monomer. For example, poly(2-isopropyl-2-oxazoline-co-2-n-butyl-2-oxazoline) may be prepared in accordance with the method of the invention by living cationic ring opening co-polymerisation of 2-isopropyl-2-oxazoline and 2-n-butyl-2-oxazoline.

In one embodiment, the polyoxazoline, polyoxazine and copolymers thereof are prepared by living cationic ring opening polymerisation of one or more monomers selected from 2-ethyl-2-oxazoline, 2-isopropyl-2-oxazoline, 2-n-propyl-2-oxazoline, 2-n-butyl-2-oxazoline, 2-N,N-diethylamino-2-oxazoline, and n-propyl-2-oxazine.

As known to those skilled in the art, the LCST of a thermoresponsive polymer used in accordance with the invention can be tailored by the choice of monomer(s) as well as ratio of co-monomers. For example, incorporating more hydrophilic monomer (e.g. 2-methyl-2-oxazoline) can increase the LCST and incorporating more hydrophobic monomer (e.g. 2-n-butyl-2-oxazoline) can decrease the LCST of the polymer.

In one embodiment, the polyoxazoline, polyoxazine and copolymers thereof are selected to exhibit a LCST between about 4° C. and about 45° C., or between about 10° C. and about 45° C., or between about 20° C. and about 38° C.

An important feature of the method of the invention is that the so-formed polyoxazoline, polyoxazine or copolymer thereof is thermoresponsive. Those skilled in the art will appreciate the thermoresponsive properties of a given polymer can be confirmed by determining the presence of an LCST as herein described.

Those skilled in the art will appreciate that living cationic ring opening polymerisation produces a propagating species having cationic charge. That propagating species is said to contain a living cation in the sense it will continue to promote polymerisation in the presence of monomer. The thermoresponsive polymer prepared in accordance with the invention therefore presents a living cation. That living cation can take part in a non-polymerisation reaction pathway. For example, that living cation may react with a nucleophilic functional of a non-monomer moiety so as to form a covalent bond between the polymer and that non-monomer moiety.

In accordance with the method of the invention, the living cation of the so formed thermoresponsive polymer reacts with a nucleophilic functional group selected from carboxylate, amino, sulphate, sulfonate, phosphate, phosphonate and thiol of the biomacromolecule. That reaction covalently couples or conjugates the so-formed thermoresponsive polymer to the biomacromolecule so as to produce the thermoresponsive polymer biomacromolecule conjugate.

One advantage of the present invention is that preparing the thermoresponsive polymer and subsequent conjugation of that polymer to the biomacromolecule can conveniently be performed in a so-called “one-pot” procedure. Such a one pot procedure enables direct conjugation of the so formed thermoresponsive polymer and the biomacromolecule without requiring isolation of the thermoresponsive polymer before conjugation with the biomacromolecule. The method in accordance with the invention therefore presents a notable synthetic advantage over conventional reaction protocols for producing biomacromolecule polymer conjugates which typically require isolation of the polymer and complex multiple synthetic steps.

While it is of course known that a living cation can react with a nucleophilic functional group, it has now surprisingly been found that producing a specific class of thermoresponsive polymer by living cationic ring opening polymerisation and reacting the living cation of the so-formed polymer with specific nucleophilic functional groups on a biomacromolecule can afford thermoresponsive polymer biomacromolecule conjugates with unique thermoresponsive and hydrogel forming properties.

Without wishing to be limited by theory, it is believed conjugation of the specific class of thermoresponsive polymer via a living cation through the specific class of nucleophilic functional groups of a biomacromolecule surprisingly provides a means for controlling and tailoring the degree of substitution of thermoresponsive polymer to the biomacromolecule. In particular, it is believed the specific nucleophilic functional groups of the biomacromolecule provide for a unique balance of reactivity with the living cation that enables excellent reactivity control over the degree of substitution. That in turn enables the conjugate to be formed such that it exhibits both an LCST and a gelation temperature and forms a hydrogel in the aqueous liquid above the LCST and gelation temperature. Conventional thermoresponsive polymer biomacromolecule conjugates will often only exhibit an LCST.

As used herein the term “degree of substitution” in the context of the conjugate refers to the percentage of available nucleophilic functional groups (NFG) of a predetermined amount within a biomacromolecule that are conjugated covalently with a thermoresponsive polymer in accordance with the invention. Formation of the conjugate can be demonstrated using DOSY-NMR, while the degree of substitution (DS) of the conjugate can be calculated based on the ratio between the peaks from the thermoresponsive polymer and the biomacromolecule in ¹H-NMR.

Further detail about the degree of substitution of conjugates prepared in accordance with the method of the invention as outlined below.

Those skilled in the art will appreciate many biomacromolecules, including those suitable for use in accordance with the present invention, comprise hydroxyl (-OH) functional groups. While hydroxyl groups can also function as a nucleophilic functional group and possibly react with a living cation, it has surprisingly been found their reactivity profile is not well-suited for preparing conjugates having the thermoresponsive and gel forming properties required according to the present invention. For example, conjugation of a thermoresponsive polymer to a biomacromolecule through hydroxyl functional groups is typically achieved by pre-reacting the -OH groups with a strong base such as NaH, which gives rise to an uncontrollable high degree of substitution and affords a conjugate not suitable for forming a hydrogel.

In one embodiment, the thermoresponsive polymer biomacromolecule conjugate prepared in accordance with the invention does not comprise thermoresponsive polymer conjugated to the biomacromolecule through a hydroxyl group.

Those skilled in the art will appreciate that conjugation of a thermoresponsive polymer to a biomacromolecule via a hydroxyl group will result in the thermoresponsive polymer being covalently coupled directly to the biomacromolecule through an oxygen atom in the form of an ether linkage.

In one embodiment, the method according to the present invention does not include reaction of the living cation with a hydroxyl group or anion thereof.

In another embodiment, the method according to the present invention does not prepare a thermoresponsive polymer biomacromolecule conjugate in which the biomacromolecule is conjugated to the thermoresponsive polymer through an ether group.

To assist with further describing the method of the invention, reference is made to Scheme 1 presented below.

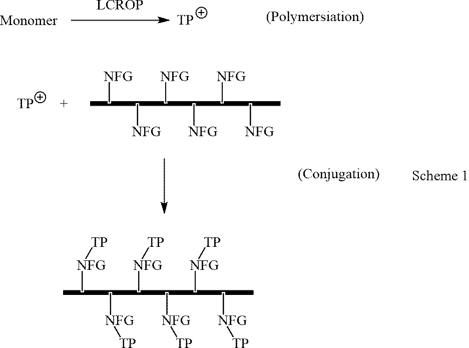

With reference to Scheme 1, in the polymerisation stage of the method according to the invention suitable monomer is subjected to living cationic ring opening polymerisation (LCROP) so as to form a thermoresponsive polymer (TP) selected from polyoxazoline, polyoxazine and copolymers thereof. Suitable monomer for undergoing LCROP are as herein described. The LCROP affords thermoresponsive polymer having a living cation (TP⁺). The so-formed TP⁺ then undergoes a conjugation step in which it reacts with a nucleophilic functional group (NFG) selected from carboxylate, amino, sulfate, sulfonate, phosphate, phosphonate and thiol of a biomacromolecule so as to covalently couple the TP to the biomacromolecule through the NFG. The TP, NFG and biomacromolecule suitable for use in accordance with the invention include those herein described.

As mentioned, the polymerisation and conjugation stages of the method according to the invention can advantageously be performed using conditions, reagents, and equipment well-known to those skilled in the art.

Those skilled in the art will appreciate that LCROP may require the use of an initiator, such as iodo/bromo acetate, methyl triflate, methyl tosylate, whereby the molecular weight of the thermoresponsive polymer can be tailored based on the molar ratio between the initiator and the monomer.

The LCROP may be conducted under anhydrous condition, often at elevated temperature between 70° C. and 200° C. to increase the rate of polymerization. For example, LCROP of oxazolines have been demonstrated at 80° C. under nitrogen or at 140° C. in microwave reactor. The LCROP is typically conducted under an inert atmosphere and in a dry aprotic solvent, such as acetonitrile, DMSO, DMF, 1,4-dioxane, or THF.

As an example only, monomer such as 2-alkyl-2-oxazoline may be mixed with an initiator, such as bromo/iodo acetate, methyl triflate or methyl sulfonate. A desired molecular weight can be achieved by adjusting the ratio between the monomer and the initiator. LCROP of the monomer is typically performed at a temperature above 70° C. Microwave irradiation can be employed to accelerate the polymerization. Anhydrous and inert conditions are typically used, such as dry aprotic solvent (e.g. acetonitrile, DMSO, DMF, 1,4-dioxane, THF) and dry atmosphere (i.e. dry nitrogen or argon). Once the LCROP is completed a dry solution of the biomacromolecule is typically added directly into the LCROP mixture for conjugation at the temperature below about 70° C.

Where the conjugation step of the method according to the invention involves reacting the living cation with a carboxylate, sulfate, sulfonate phosphate or phosphonate nucleophilic functional group, those groups will generally be provided in the form of a salt. For example, those nucleophilic functional groups may be provided in the form of a sodium, potassium, phosphonium and ammonium salt.

In one embodiment, the carboxylate, sulfate, sulfonate phosphate and phosphonate nucleophilic functional groups are provided in the form of a tetraalkyl or tetraaryl phosphonium or ammonium salt.

Where the living cation is reacted with an amino nucleophilic functional group, the amino group may be a primary, secondary, or tertiary amino group.

In one embodiment, the amino nucleophilic functional group is a primary or a secondary amino group.

Once the thermoresponsive polymer biomacromolecule conjugate is formed, it will generally be isolated from the reaction mixture using techniques known in the art. For example, the so-formed conjugate may be isolated and purified using dialysis and/or precipitation in a non-solvent.

The method according to the present invention provides an effective and efficient means to produce conjugates that exhibit both an LCST and gelation temperature in an aqueous liquid and can form a hydrogel in the aqueous liquid above the gelation temperature.

Conjugates produced in accordance with the invention are advantageously suitable for use in biomedical hydrogel applications.

Evaluating the thermoresponsive and gel forming properties of conjugates produced in accordance with the invention can be achieved simply by dissolving the conjugates in an aqueous liquid and evaluating that liquid for a gel formation above the LCST of the conjugate according to protocols well-known to those skilled in the art. For example, a rheometer with a temperature sweep can be used to assess the gel formation. When the storage modulus (G′) is lower than the loss modulus (G″), the state of the aqueous liquid comprising the conjugate will be in a liquid-like state. The liquid-to-gel phase transition occurs when storage modulus (G′) increases to the same value as the loss modulus (G″), which is expressed by the rheometer through the tangent δ or G″/G′ ratio being equal to 1. The temperature at which such liquid-to-gel phase transition occurs is defined as the gelation temperature of the hydrogel. Once formed, the presence or persistence of the hydrogel can also be determined using a rheometer when tangent δ ≤ 1 or G′>G″.

In one embodiment, an aqueous liquid comprising the thermoresponsive polymer biomacromolecule conjugate prepared in accordance with the method of the invention exhibits a tangent δ ≤ 1 at a temperature ≤ 37° C., as measured by a rheometer.

In a further embodiment, an aqueous liquid comprising the thermoresponsive polymer biomacromolecule conjugate prepared in accordance with the method of the invention exhibits (i) a tangent δ > 1 at a temperature up to about 25° C., or about 26° C., or about 27° C., and (ii) a tangent δ ≤ 1 at a temperature ≤ 37° C., as measured by a rheometer.

In another embodiment, the thermoresponsive polymer biomacromolecule conjugate prepared in accordance with the method of the invention forms in a aqueous liquid a hydrogel that exhibits a storage modulus (G′) that is ≥ 100 Pa at ≥37° C., as measured by a rheometer.

In one application of forming a hydrogel, the method according to the invention may further comprise isolating the so-formed thermoresponsive polymer biomacromolecule conjugate and dissolving it in an aqueous liquid.

Reference herein to an “aqueous liquid” is intended to mean a liquid comprising at least about 50%, or about 60%, or about 70%, or about 80%, or about 90%, or about 95%, water. The aqueous liquid may comprise one or more water soluble reagents, for example, water soluble solvents, excipients and/or salts.

In one embodiment, the aqueous liquid contains one or more of surfactant (e.g. poloxamer, polysorbate), amino acids (e.g. arginine, lysine), sugars (e.g. glucose, trehalose), chelating agents (e.g. EDTA, citric acid), buffer/stabilizing salt (e.g. potassium phosphate, sodium sulphate), stabilizing polymer (e.g. PEG, dextran, PVA) and sodium chloride.

In one embodiment, the aqueous liquid is a phosphate-buffered saline liquid.

In a further embodiment, the aqueous liquid is saline solution, for example an isotonic saline solution.

Once dissolved in the aqueous liquid, the conjugate prepared in accordance with the method of the invention will exhibit both a LCST and gelation temperature. The LCST and gelation temperature can be readily determined as herein described.

It has now been found that parameters such as the degree substitution of thermoresponsive polymer conjugated to the biomacromolecule, molecular weight of the thermoresponsive polymer and the biomacromolecule (including the ratio between those two features) play a role in the ability of a given conjugate to form a hydrogel.

For example, while HA substituted with poly(2-isopropyl-2-oxazoline-co-2-n-butyl-2-oxazoline) at about 60% has an LCST, it does not have a gelation temperature and consequently does not form a hydrogel. Rather such a conjugate forms a precipitate (i.e. micro/nanoparticles) instead of hydrogel above its LCST.

To date techniques for preparing thermoresponsive polymer biomacromolecule conjugates have either been complex or offered little or no control over reaction parameters such as the degree of substitution and conjugation of the biomacromolecule to the thermoresponsive polymer. The method in accordance with the present invention is advantageously not only simple but also offers excellent reaction control thereby enabling the effective and efficient production of conjugates capable of forming hydrogels. Furthermore, the hydrogels can be produced so as to exhibit excellent physical properties, for example having a storage modulus (G′) of greater than 100 Pa at 37° C., as measured using a rheometer.

The gel properties of the so-formed hydrogels can be particularly important in certain biomedical applications where relatively firm gels might be required. Being able to control and adjust the physical form of the gel can also advantageously influence the rate of diffusion of a reagent, such as a drug, out of the hydrogel matrix. In some applications it can also be advantageous for the hydrogel to have similar mechanical properties to body tissue in which the hydrogel has been implanted.

The method according to the invention advantageously provides means to control the molecular composition and architecture of the so-formed conjugates that in turn enables control over the gel properties of a hydrogel formed using the conjugates.

The present invention also provides a method of forming a hydrogel, the method comprising: providing an aqueous liquid solution of thermoresponsive polymer biomacromolecule conjugate prepared in accordance with the method of the invention, the thermoresponsive polymer biomacromolecule conjugate exhibiting in the aqueous liquid a gelation temperature; and raising the temperature of the aqueous liquid comprising the thermoresponsive polymer biomacromolecule conjugate above the gelation temperature so as to promote formation of the hydrogel.

Without wishing to be limited by theory, it is believed formation of a hydrogel from an aqueous liquid of thermoresponsive polymer biomacromolecule conjugate in accordance with the invention is influenced by the interplay between various parameters. Those parameters include the LCST of the thermoresponsive polymer, the molecular weight of the thermoresponsive polymer and the biomacromolecule (including the ratio between those two components), the degree of substitution and molecular distribution of the thermoresponsive polymer on the biomacromolecule, and the concentration of the conjugate in an aqueous solution.

The method of forming the conjugates in accordance with the invention advantageously enables excellent control of most of those parameters.

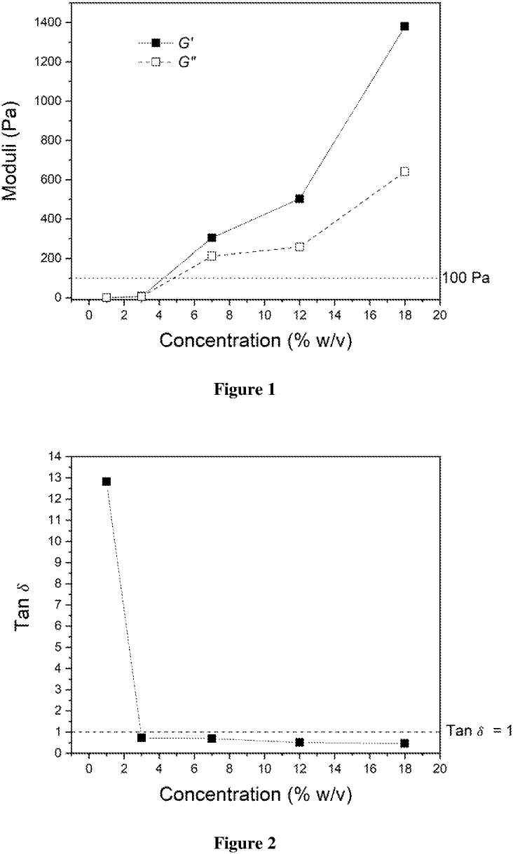

The method according to the present invention is very well suited to controlling the degree of substitution (DS) of the thermoresponsive polymer on a biomacromolecule. Without wishing to be limited by theory, that DS seems to play a role in the hydrogel formation. For example, with reference to FIG. 4, the conjugate comprising of HA with 500 kDa (HA₅₀₀) and poly(2-isopropyl-2-oxazoline-co-2-n-butyl-2-oxazoline) with 13 kDa forms a hydrogel at a DS ranging between about 2.5% and about 28% at 18% w/v concentration (Conjugates 4 - 8). However, higher DS (e.g. about 60%) leads to precipitation or no hydrogel formation (Conjugate 9). Lower concentration of the conjugate (e.g. about 7% w/v) narrows the hydrogel forming DS range further between about 5.9% and about 16% (Conjugates 5 - 7). It is surprising that a maximum of G′ at 37° C. seems to be achieved at certain DSₘₐₓ for the conjugates. The experimental data using the same poly(2-isopropyl-2-oxazoline-co-2-n-butyl-2-oxazoline) composition also shows that higher concentration of the 50 kDa HA conjugate increases the modulus (G′) of the resultant hydrogel and decrease the gelation temperature (see FIGS. 1 and 2). In contrast, lower molecular weight of HA (conjugate of 20 kDa HA with 13 kDa polyoxazoline) did not exhibit hydrogel formation at 37° C. (Conjugate 1). Nevertheless, if the gelation temperature is found to be higher than 37° C. and needs to be decreased to obtain a hydrogel at 37° C. (tan δ < 1), the LCST of the thermoresponsive polymer can be decreased further by incorporating more hydrophobic monomer in its molecular structure. Decreasing the LCST of the thermoresponsive polymer is able to increase the storage modulus (G′) at 37° C. at a particular DS of the conjugate and its concentration in an aqueous liquid (Conjugate 11).

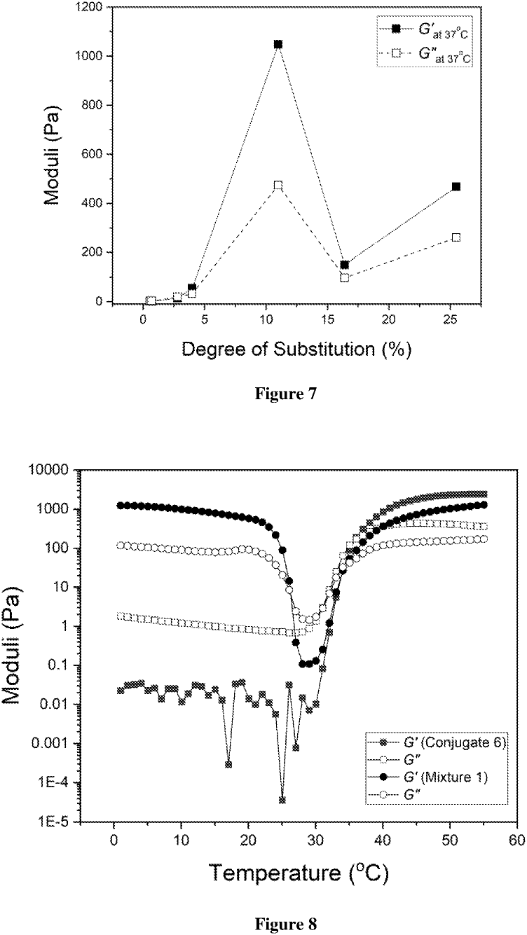

With reference to FIG. 7, the conjugate derived from a pre-aminated porcine gelatine (bloom 300) and poly(2-isopropyl-2-oxazoline-co-2-n-butyl-2-oxazoline) forms a hydrogel at concentration ≥ 20% w/v only (no gelation is observed at 10% or 15% w/v).

Without wishing to be limited by theory, for a number of conjugates hydrogel formation seems to be optimal at a DS ranging between about 5% and about 25%.

In one embodiment, the living cation is reacted with the nucleophilic functional groups of the biomacromolecule to afford the conjugate having a degree of substitution ranging from 1% to about 50%, or from about 5% to about 30%, or from about 5% to about 25%, or from about 10% to about 50%, or from about 10% to about 30%, or from about 10% to about 25%.

When forming a hydrogel in accordance with the method of the invention, the conjugate will generally present in the aqueous liquid solution at concentration ranging from about 2%v/w to about 40% v/w.

In one embodiment, the aqueous liquid solution comprises about 2%v/w to about 40% v/w, or about 5%v/w to about 40% v/w, or about 5%v/w to about 30% v/w of the conjugate.

Without wishing to be limited by theory, is believed that the thermoresponsive polymer used in accordance with the invention imparts a LCST to the so-formed conjugate, with the conjugate itself undergoing a physical transformation in transitioning through its LCST. As with the thermoresponsive polymer, when the so-formed conjugate transitions through the LCST it also undergoes a transformation from exhibiting hydrophilic character to exhibiting hydrophobic character, with that process being reversible. That LCST-mediated transition enables the thermoresponsive polymer domains of the conjugate to self-assemble. Molecular distribution of the thermoresponsive polymer across the biomacromolecule structure in the form of the conjugate is believed to influence the ability of the conjugate to exhibit a gelation temperature and hydrogel formation. The practical effect of that molecular distribution is for the most part determined by the degree of substitution and the molecular weight of the thermoresponsive polymer and the biomacromolecule. Surprisingly, obtaining a suitable combination between the LCST and molecular distribution of the conjugate advantageously enables the conjugate to reversibly transition from being in solution to forming a hydrogel upon transitioning through the gelation temperature. For example, when in an aqueous liquid the conjugate can present in the form of a free-flowing injectable liquid below the gelation temperature and transition into a hydrogel above the gelation temperature.

In one embodiment, the gelation temperature of the conjugate in the aqueous liquid ranges from about 4° C. to about 45° C., or about 20° C. to about 38° C., or about 30° C. to about 37° C.

As discussed herein, above the gelation temperature the aqueous solution comprising the conjugate in accordance with the invention transitions into a hydrogel. Those skilled in the art are familiar with the concept of hydrogel formation and can readily identify the onset of hydrogel formation.

According to the present invention, a hydrogel is intended to mean an aqueous liquid comprising the conjugate which has a tangent delta less than 1 (i.e., tan δ = G″/G′ < 1) as measured using a rheometer. It will be appreciated the tangent delta of that aqueous solution will be measured at a temperature that is at least above the gelation temperature.

In a one embodiment, the so-formed hydrogel exhibit tangent δ or G″/G′ ≤1 at 37° C., as measured by rheometer.

In another embodiment, the so-formed hydrogel has a storage modulus (G′) of greater than about 100 Pa, for example, ranging from about 100 Pa to about 40,000 Pa, or from about 100 Pa to about 20,000 Pa, or from about 100 Pa to about 10000 Pa, or from about 100 Pa to about 5000 Pa, or about 200 Pa to about 40,000 Pa, or from about 200 Pa to about 20,000 Pa, or from about 200 Pa to about 10000 Pa, or from about 200 Pa to about 5000 Pa.

In a further embodiment, the storage modulus (G′) of the hydrogel referenced herein is that measured at a temperature ranging from about 20° C. to about 45° C., or from about 25° C. to about 37° C. or from about 30° C. to about 37° C., or at about 37° C.

Depending on the intended application of hydrogels that can be produced in accordance with the invention, it may be desirable they be formulated to incorporate an agent within the so-formed hydrogel matrix. That agent can be incorporated within the hydrogel matrix for the purpose of diffusing out of the hydrogel matrix over time, for example, a hydrogel loaded with an agent could be implanted into a subject for the purpose of that agent diffusing out from the hydrogel matrix into the subject over time.

Adjusting the gel properties of a hydrogel formed using the conjugate can advantageously enable the release profile of an agent contained within the hydrogel to be tailored as a required.

An agent can be readily incorporated within the hydrogel simply by combining the agent with the aqueous liquid when forming the solution of the conjugate. For example, the agent can be dispersed in the aqueous solution of the conjugate at a temperature below the gelation temperature. Above the gelation temperature the aqueous solution transitions into a hydrogel thereby capturing the agent within the hydrogel matrix.

There is no particular limitation on the type of agent that can be incorporated within the hydrogel.

For example, the agent may be a biologically active agent. The agent may be in the form of microparticles and/or nanoparticles. Those microparticles or nanoparticles may themselves be biologically active agents.

By the agent being “biologically active” means it is intended for use in the diagnosis, cure, mitigation, treatment, prevention or modification of a state in a biological system. For example, the agent may be a drug that is used to therapeutically to treat or prevent a disease state in humans or other animal species.

In one embodiment, the agent is a biologically active agent.

In another embodiment, the agent is in the form of microparticles and/or nanoparticles

Examples of biologically active agents include, but are not limited to, antibiotics, antimicrobial agents, anti-viral agents, anaesthetics, steroidal agents, anti-inflammatory agents, anti-neoplastic agents, antigens, vaccines, antibodies, growth factors, decongestants, antihypertensives, sedatives, birth control agents, progestational agents, anti-cholinergics, analgesics, anti-depressants, anti-psychotics, β-adrenergic blocking agents, diuretics, cardiovascular active agents, vasoactive agents, non-steroidal anti-inflammatory agents, nutritional agents and prostaglandin.

As used herein, the term “alkyl”, used either alone or in compound words denotes straight chain, branched or cyclic alkyl, for example C₁₋₄₀ alkyl, or C₁₋₂₀ or C₁₋₁₀. Examples of straight chain and branched alkyl include methyl, ethyl, n-propyl, isopropyl, n-butyl, sec-butyl, t-butyl, n-pentyl, 1,2-dimethylpropyl, 1,1-dimethyl-propyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethyl-pentyl, 1,2,3-trimethylbutyl, 1,1,2-trimethylbutyl, 1,1,3-trimethylbutyl, octyl, 6-methylheptyl, 1-methylheptyl, 1,1,3,3-tetramethylbutyl, nonyl, 1-, 2-, 3-, 4-, 5-, 6- or 7-methyloctyl, 1-, 2-, 3-, 4- or 5-ethylheptyl, 1-, 2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5-or 6-propylnonyl, 1-, 2-, 3- or 4-butyloctyl, 1-2-pentylheptyl, tridecyl, tetradecyl, pentadecyl, hexadecyl, heptadecyl, octadecyl, nonoadecyl, eicosyl and the like. Examples of cyclic alkyl include mono- or polycyclic alkyl groups such as cyclopropyl, cyclobutyl, cyclopentyl, cyclohexyl, cycloheptyl, cyclooctyl, cyclononyl, cyclodecyl and the like. Where an alkyl group is referred to generally as “propyl”, butyl” etc, it will be understood that this can refer to any of straight, branched and cyclic isomers where appropriate. An alkyl group may be optionally substituted by one or more optional substituents as herein defined.

In this specification “optionally substituted” is taken to mean that a group may or may not be substituted or fused (so as to form a condensed polycyclic group) with one, two, three or more of organic and inorganic groups (i.e. the optional substituent) including those selected from: alkyl, alkenyl, alkynyl, carbocyclyl, aryl, heterocyclyl, heteroaryl, acyl, aralkyl, alkaryl, alkheterocyclyl, alkheteroaryl, alkcarbocyclyl, halo, haloalkyl, haloalkenyl, haloalkynyl, haloaryl, halocarbocyclyl, haloheterocyclyl, haloheteroaryl, haloacyl, haloaryalkyl, hydroxy, hydroxyalkyl, hydroxyalkenyl, hydroxyalkynyl, hydroxycarbocyclyl, hydroxyaryl, hydroxyheterocyclyl, hydroxyheteroaryl, hydroxyacyl, hydroxyaralkyl, alkoxyalkyl, alkoxyalkenyl, alkoxyalkynyl, alkoxycarbocyclyl, alkoxyaryl, alkoxyheterocyclyl, alkoxyheteroaryl, alkoxyacyl, alkoxyaralkyl, alkoxy, alkenyloxy, alkynyloxy, aryloxy, carbocyclyloxy, aralkyloxy, heteroaryloxy, heterocyclyloxy, acyloxy, haloalkoxy, haloalkenyloxy, haloalkynyloxy, haloaryloxy, halocarbocyclyloxy, haloaralkyloxy, haloheteroaryloxy, haloheterocyclyloxy, haloacyloxy, nitro, nitroalkyl, nitroalkenyl, nitroalkynyl, nitroaryl, nitroheterocyclyl, nitroheteroayl, nitrocarbocyclyl, nitroacyl, nitroaralkyl, amino (NH₂), alkylamino, dialkylamino, alkenylamino, alkynylamino, arylamino, diarylamino, aralkylamino, diaralkylamino, acylamino, diacylamino, heterocyclamino, heteroarylamino, carboxy, carboxyester, amido, alkylsulphonyloxy, arylsulphenyloxy, alkylsulphenyl, arylsulphenyl, thio, alkylthio, alkenylthio, alkynylthio, arylthio, aralkylthio, carbocyclylthio, heterocyclylthio, heteroarylthio, acylthio, sulfoxide, sulfonyl, sulfonamide, aminoalkyl, aminoalkenyl, aminoalkynyl, aminocarbocyclyl, aminoaryl, aminoheterocyclyl, aminoheteroaryl, aminoacyl, aminoaralkyl, thioalkyl, thioalkenyl, thioalkynyl, thiocarbocyclyl, thioaryl, thioheterocyclyl, thioheteroaryl, thioacyl, thioaralkyl, carboxyalkyl, carboxyalkenyl, carboxyalkynyl, carboxycarbocyclyl, carboxyaryl, carboxyheterocyclyl, carboxyheteroaryl, carboxyacyl, carboxyaralkyl, carboxyesteralkyl, carboxyesteralkenyl, carboxyesteralkynyl, carboxyestercarbocyclyl, carboxyesteraryl, carboxyesterheterocyclyl, carboxyesterheteroaryl, carboxyesteracyl, carboxyesteraralkyl, amidoalkyl, amidoalkenyl, amidoalkynyl, amidocarbocyclyl, amidoaryl, amidoheterocyclyl, amidoheteroaryl, amidoacyl, amidoaralkyl, formylalkyl, formylalkenyl, formylalkynyl, formylcarbocyclyl, formylaryl, formylheterocyclyl, formylheteroaryl, formylacyl, formylaralkyl, acylalkyl, acylalkenyl, acylalkynyl, acylcarbocyclyl, acylaryl, acylheterocyclyl, acylheteroaryl, acylacyl, acylaralkyl, sulfoxidealkyl, sulfoxidealkenyl, sulfoxidealkynyl, sulfoxidecarbocyclyl, sulfoxidearyl, sulfoxideheterocyclyl, sulfoxideheteroaryl, sulfoxideacyl, sulfoxidearalkyl, sulfonylalkyl, sulfonylalkenyl, sulfonylalkynyl, sulfonylcarbocyclyl, sulfonylaryl, sulfonylheterocyclyl, sulfonylheteroaryl, sulfonylacyl, sulfonylaralkyl, sulfonamidoalkyl, sulfonamidoalkenyl, sulfonamidoalkynyl, sulfonamidocarbocyclyl, sulfonamidoaryl, sulfonamidoheterocyclyl, sulfonamidoheteroaryl, sulfonamidoacyl, sulfonamidoaralkyl, nitroalkyl, nitroalkenyl, nitroalkynyl, nitrocarbocyclyl, nitroaryl, nitroheterocyclyl, nitroheteroaryl, nitroacyl, nitroaralkyl, cyano, sulfate and phosphate groups.

Optional substituents may include alkyl (e.g. C₁₋₆ alkyl such as methyl, ethyl, propyl, butyl, cyclopropyl, cyclobutyl, cyclopentyl or cyclohexyl), hydroxyalkyl (e.g. hydroxymethyl, hydroxyethyl, hydroxypropyl), alkoxyalkyl (e.g. methoxymethyl, methoxyethyl, methoxypropyl, ethoxymethyl, ethoxyethyl, ethoxypropyl etc) alkoxy (e.g. C₁₋₆ alkoxy such as methoxy, ethoxy, propoxy, butoxy, cyclopropoxy, cyclobutoxy), halo, trifluoromethyl, trichloromethyl, tribromomethyl, hydroxy, phenyl (which itself may be further substituted e.g., by C₁₋₆ alkyl, halo, hydroxy, hydroxyC₁₋₆ alkyl, C₁₋₆ alkoxy, haloC₁₋ ₆alkyl, cyano, nitro OC(O)C₁₋₆ alkyl, and amino), benzyl (wherein benzyl itself may be further substituted e.g., by C₁₋₆ alkyl, halo, hydroxy, hydroxyC₁₋₆alkyl, C₁₋₆ alkoxy, haloC₁₋₆ alkyl, cyano, nitro OC(O)C₁₋₆ alkyl, and amino), phenoxy (wherein phenyl itself may be further substituted e.g., by C₁₋₆ alkyl, halo, hydroxy, hydroxyC₁₋₆ alkyl, C₁₋₆ alkoxy, haloC₁₋ ₆ alkyl, cyano, nitro OC(O)C₁₋₆ alkyl, and amino), benzyloxy (wherein benzyl itself may be further substituted e.g., by C₁₋₆ alkyl, halo, hydroxy, hydroxyC₁₋₆ alkyl, C₁₋₆ alkoxy, haloC₁₋ ₆ alkyl, cyano, nitro OC(O)C₁₋₆ alkyl, and amino), amino, alkylamino (e.g. C₁₋₆ alkyl, such as methylamino, ethylamino, propylamino etc), dialkylamino (e.g. C₁₋₆ alkyl, such as dimethylamino, diethylamino, dipropylamino), acylamino (e.g. NHC(O)CH₃), phenylamino (wherein phenyl itself may be further substituted e.g., by C₁₋₆ alkyl, halo, hydroxy hydroxyC₁₋₆ alkyl, C₁₋₆ alkoxy, haloC₁₋₆ alkyl, cyano, nitro OC(O)C₁₋₆ alkyl, and amino), nitro, formyl, -C(O)-alkyl (e.g. C₁₋₆ alkyl, such as acetyl), O-C(O)-alkyl (e.g. C₁₋₆alkyl, such as acetyloxy), benzoyl (wherein the phenyl group itself may be further substituted e.g., by C₁₋₆ alkyl, halo, hydroxy hydroxyC₁₋₆ alkyl, C₁₋₆ alkoxy, haloC₁₋₆ alkyl, cyano, nitro OC(O)C₁₋₆alkyl, and amino), replacement of CH₂ with C=O, CO₂H, CO₂alkyl (e.g. C₁₋₆ alkyl such as methyl ester, ethyl ester, propyl ester, butyl ester), CO₂phenyl (wherein phenyl itself may be further substituted e.g., by C₁₋₆ alkyl, halo, hydroxy, hydroxyl C₁₋₆ alkyl, C₁₋₆ alkoxy, halo C₁₋₆ alkyl, cyano, nitro OC(O)C₁₋₆ alkyl, and amino), CONH₂, CONHphenyl (wherein phenyl itself may be further substituted e.g., by C₁₋₆ alkyl, halo, hydroxy, hydroxyl C₁₋₆ alkyl, C₁₋₆ alkoxy, halo C₁₋₆ alkyl, cyano, nitro OC(O)C₁₋₆ alkyl, and amino), CONHbenzyl (wherein benzyl itself may be further substituted e.g., by C₁₋₆ alkyl, halo, hydroxy hydroxyl C₁₋₆ alkyl, C₁₋₆ alkoxy, halo C₁₋₆ alkyl, cyano, nitro OC(O)C₁₋₆ alkyl, and amino), CONHalkyl (e.g. C₁₋₆ alkyl such as methyl ester, ethyl ester, propyl ester, butyl amide) CONHdialkyl (e.g. C₁₋₆ alkyl) aminoalkyl (e.g., HN C₁₋₆ alkyl-, C₁₋₆alkylHN-C₁₋₆ alkyl- and (C₁₋₆ alkyl)₂N-C₁₋₆ alkyl-), thioalkyl (e.g., HS C₁₋₆ alkyl-), carboxyalkyl (e.g., HO₂CC₁₋₆ alkyl-), carboxyesteralkyl (e.g., C₁₋₆ alkylO₂CC₁₋₆ alkyl-), amidoalkyl (e.g., H₂N(O)CC₁₋₆ alkyl-, H(C₁₋₆ alkyl)N(O)CC₁₋₆ alkyl-), formylalkyl (e.g., OHCC₁₋₆alkyl-), acylalkyl (e.g., C₁₋₆ alkyl(O)CC₁₋₆ alkyl-), nitroalkyl (e.g., O₂NC₁₋₆ alkyl-), sulfoxidealkyl (e.g., R³(O)SC₁₋₆ alkyl, such as C₁₋₆ alkyl(O)SC₁₋₆ alkyl-), sulfonylalkyl (e.g., R³(O)₂SC₁₋₆ alkyl- such as C₁₋₆ alkyl(O)₂SC₁₋₆ alkyl-), sulfonamidoalkyl (e.g., ₂HRN(O)SC₁₋₆ alkyl, H(C₁₋₆ alkyl)N(O)SC₁₋₆ alkyl-).

As used herein, term “alkenyl” denotes groups formed from straight chain, branched or cyclic hydrocarbon residues containing at least one carbon to carbon double bond including ethylenically mono-, di- or polyunsaturated alkyl or cycloalkyl groups as previously defined, for example C₂₋₄₀ alkenyl, or C₂₋₂₀ or C₂₋₁₀. Thus, alkenyl is intended to include propenyl, butylenyl, pentenyl, hexaenyl, heptaenyl, octaenyl, nonaenyl, decenyl, undecenyl, dodecenyl, tridecenyl, tetradecenyl, pentadecenyl, hexadecenyl, heptadecenyl, octadecenyl, nondecenyl, eicosenyl hydrocarbon groups with one or more carbon to carbon double bonds. Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, bicycloheptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl. An alkenyl group may be optionally substituted by one or more optional substituents as herein defined.

As used herein the term “alkynyl” denotes groups formed from straight chain, branched or cyclic hydrocarbon residues containing at least one carbon-carbon triple bond including ethylenically mono-, di- or polyunsaturated alkyl or cycloalkyl groups as previously defined, for example, C₂₋₄₀ alkenyl, or C₂₋₂₀ or C₂₋₁₀. Thus, alkynyl is intended to include propynyl, butylynyl, pentynyl, hexaynyl, heptaynyl, octaynyl, nonaynyl, decynyl, undecynyl, dodecynyl, tridecynyl, tetradecynyl, pentadecynyl, hexadecynyl, heptadecynyl, octadecynyl, nondecynyl, eicosynyl hydrocarbon groups with one or more carbon to carbon triple bonds. Examples of alkynyl include ethynyl, 1-propynyl, 2-propynyl, and butynyl isomers, and pentynyl isomers. An alkynyl group may be optionally substituted by one or more optional substituents as herein defined.

An alkenyl group may comprise a carbon to carbon triple bond and an alkynyl group may comprise a carbon to carbon double bond (i.e. so called ene-yne or yne-ene groups).

As used herein, the term “aryl” (or “carboaryl)” denotes any of single, polynuclear, conjugated and fused residues of aromatic hydrocarbon ring systems. Examples of aryl include phenyl, biphenyl, terphenyl, quaterphenyl, naphthyl, tetrahydronaphthyl, anthracenyl, dihydroanthracenyl, benzanthracenyl, dibenzanthracenyl, phenanthrenyl, fluorenyl, pyrenyl, idenyl, azulenyl, chrysenyl. Preferred aryl include phenyl and naphthyl. An aryl group may be optionally substituted by one or more optional substituents as herein defined.

As used herein, the terms “alkylene”, “alkenylene”, and “arylene” are intended to denote the divalent forms of “alkyl”, “alkenyl”, and “aryl”, respectively, as herein defined.

The term “halogen” (“halo”) denotes fluorine, chlorine, bromine or iodine (fluoro, chloro, bromo or iodo). Preferred halogens are chlorine, bromine or iodine.

The term “carbocyclyl” includes any of non-aromatic monocyclic, polycyclic, fused or conjugated hydrocarbon residues, preferably C₃₋₂₀ (e.g. C₃₋₁₀ or C₃₋₈). The rings may be saturated, e.g. cycloalkyl, or may possess one or more double bonds (cycloalkenyl) and/or one or more triple bonds (cycloalkynyl). Particularly preferred carbocyclyl moieties are 5-6-membered or 9-10 membered ring systems. Suitable examples include cyclopropyl, cyclobutyl, cyclopentyl, cyclohexyl, cycloheptyl, cyclooctyl, cyclononyl, cyclodecyl, cyclopentenyl, cyclohexenyl, cyclooctenyl, cyclopentadienyl, cyclohexadienyl, cyclooctatetraenyl, indanyl, decalinyl and indenyl.

The term “heterocyclyl” when used alone or in compound words includes any of monocyclic, polycyclic, fused or conjugated hydrocarbon residues, preferably C₃₋₂₀ (e.g. C₃₋ ₁₀ or C₃₋₈) wherein one or more carbon atoms are replaced by a heteroatom so as to provide a non-aromatic residue. Suitable heteroatoms include O, N, S, P and Se, particularly O, N and S. Where two or more carbon atoms are replaced, this may be by two or more of the same heteroatom or by different heteroatoms. The heterocyclyl group may be saturated or partially unsaturated, i.e. possess one or more double bonds. Particularly preferred heterocyclyl are 5-6 and 9-10 membered heterocyclyl. Suitable examples of heterocyclyl groups may include azridinyl, oxiranyl, thiiranyl, azetidinyl, oxetanyl, thietanyl, 2H-pyrrolyl, pyrrolidinyl, pyrrolinyl, piperidyl, piperazinyl, morpholinyl, indolinyl, imidazolidinyl, imidazolinyl, pyrazolidinyl, thiomorpholinyl, dioxanyl, tetrahydrofuranyl, tetrahydropyranyl, tetrahydropyrrolyl, tetrahydrothiophenyl, pyrazolinyl, dioxalanyl, thiazolidinyl, isoxazolidinyl, dihydropyranyl, oxazinyl, thiazinyl, thiomorpholinyl, oxathianyl, dithianyl, trioxanyl, thiadiazinyl, dithiazinyl, trithianyl, azepinyl, oxepinyl, thiepinyl, indenyl, indanyl, 3H-indolyl, isoindolinyl, 4H-quinolazinyl, chromenyl, chromanyl, isochromanyl, pyranyl and dihydropyranyl.

The term “heteroaryl” includes any of monocyclic, polycyclic, fused or conjugated hydrocarbon residues, wherein one or more carbon atoms are replaced by a heteroatom so as to provide an aromatic residue. Preferred heteroaryl have 3-20 ring atoms, e.g. 3-10. Particularly preferred heteroaryl are 5-6 and 9-10 membered bicyclic ring systems. Suitable heteroatoms include, O, N, S, P and Se, particularly O, N and S. Where two or more carbon atoms are replaced, this may be by two or more of the same heteroatom or by different heteroatoms. Suitable examples of heteroaryl groups may include pyridyl, pyrrolyl, thienyl, imidazolyl, furanyl, benzothienyl, isobenzothienyl, benzofuranyl, isobenzofuranyl, indolyl, isoindolyl, pyrazolyl, pyrazinyl, pyrimidinyl, pyridazinyl, indolizinyl, quinolyl, isoquinolyl, phthalazinyl, 1,5-naphthyridinyl, quinozalinyl, quinazolinyl, quinolinyl, oxazolyl, thiazolyl, isothiazolyl, isoxazolyl, triazolyl, oxadialzolyl, oxatriazolyl, triazinyl, and furazanyl.

The term “acyl” either alone or in compound words denotes a group containing the agent C=O (and not being a carboxylic acid, ester or amide) Preferred acyl includes C(O)-R^{x}, wherein R^{x} is hydrogen or an alkyl, alkenyl, alkynyl, aryl, heteroaryl, carbocyclyl, or heterocyclyl residue. Examples of acyl include formyl, straight chain or branched alkanoyl (e.g. C₁₋₂₀) such as, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl and icosanoyl; cycloalkylcarbonyl such as cyclopropylcarbonyl cyclobutylcarbonyl, cyclopentylcarbonyl and cyclohexylcarbonyl; aroyl such as benzoyl, toluoyl and naphthoyl; aralkanoyl such as phenylalkanoyl (e.g. phenylacetyl, phenylpropanoyl, phenylbutanoyl, phenylisobutylyl, phenylpentanoyl and phenylhexanoyl) and naphthylalkanoyl (e.g. naphthylacetyl, naphthylpropanoyl and naphthylbutanoyl]; aralkenoyl such as phenylalkenoyl (e.g. phenylpropenoyl, phenylbutenoyl, phenylmethacryloyl, phenylpentenoyl and phenylhexenoyl and naphthylalkenoyl (e.g. naphthylpropenoyl, naphthylbutenoyl and naphthylpentenoyl); aryloxyalkanoyl such as phenoxyacetyl and phenoxypropionyl; arylthiocarbamoyl such as phenylthiocarbamoyl; arylglyoxyloyl such as phenylglyoxyloyl and naphthylglyoxyloyl; arylsulfonyl such as phenylsulfonyl and napthylsulfonyl; heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; heterocyclicalkenoyl such as heterocyclicpropenoyl, heterocyclicbutenoyl, heterocyclicpentenoyl and heterocyclichexenoyl; and heterocyclicglyoxyloyl such as thiazolyglyoxyloyl and thienylglyoxyloyl. The R^{x} residue may be optionally substituted as described herein.

The term “sulfoxide”, either alone or in a compound word, refers to a group -S(O)R^{y} wherein R^{y} is selected from hydrogen, alkyl, alkenyl, alkynyl, aryl, heteroaryl, heterocyclyl, carbocyclyl, and aralkyl. Examples of preferred R^{y} include C₁₋₂₀alkyl, phenyl and benzyl.

The term “sulfonyl”, either alone or in a compound word, refers to a group S(O)₂-R^{y}, wherein R^{y} is selected from hydrogen, alkyl, alkenyl, alkynyl, aryl, heteroaryl, heterocyclyl, carbocyclyl and aralkyl. Examples of preferred R^{y} include C₁₋₂₀alkyl, phenyl and benzyl.

The term “sulfonamide”, either alone or in a compound word, refers to a group S(O)NR^{y}R^{y} wherein each R^{y} is independently selected from hydrogen, alkyl, alkenyl, alkynyl, aryl, heteroaryl, heterocyclyl, carbocyclyl, and aralkyl. Examples of preferred R^{y} include C₁₋₂₀alkyl, phenyl and benzyl. In a preferred embodiment at least one R^{y} is hydrogen. In another form, both R^{y} are hydrogen.

The term, “amino” is used here in its broadest sense as understood in the art and includes groups of the formula NR^{A}R^{B} wherein R^{A} and R^{B} may be any independently selected from hydrogen, alkyl, alkenyl, alkynyl, aryl, carbocyclyl, heteroaryl, heterocyclyl, aralkyl, and acyl. R^{A} and R^{B}, together with the nitrogen to which they are attached, may also form a monocyclic, or polycyclic ring system e.g. a 3-10 membered ring, particularly, 5-6 and 9-10 membered systems. Examples of “amino” include NH₂, NHalkyl (e.g. C₁₋ ₂₀alkyl), NHaryl (e.g. NHphenyl), NHaralkyl (e.g. NHbenzyl), NHacyl (e.g. NHC(O)C₁₋ ₂₀alkyl, NHC(O)phenyl), Nalkylalkyl (wherein each alkyl, for example C₁₋₂₀, may be the same or different) and 5 or 6 membered rings, optionally containing one or more same or different heteroatoms (e.g. O, N and S).

The term “amido” is used here in its broadest sense as understood in the art and includes groups having the formula C(O)NR^{A}R^{B}, wherein R^{A} and R^{B} are as defined as above. Examples of amido include C(O)NH₂, C(O)NHalkyl (e.g. C₁₋₂₀alkyl), C(O)NHaryl (e.g. C(O)NHphenyl), C(O)NHaralkyl (e.g. C(O)NHbenzyl), C(O)NHacyl (e.g. C(O)NHC(O)C₁₋ ₂₀alkyl, C(O)NHC(O)phenyl), C(O)Nalkylalkyl (wherein each alkyl, for example C₁₋₂₀, may be the same or different) and 5 or 6 membered rings, optionally containing one or more same or different heteroatoms (e.g. O, N and S).

The term “carboxy ester” is used here in its broadest sense as understood in the art and includes groups having the formula CO₂R^{z}, wherein R^{z} may be selected from groups including alkyl, alkenyl, alkynyl, aryl, carbocyclyl, heteroaryl, heterocyclyl, aralkyl, and acyl. Examples of carboxy ester include CO₂C₁₋₂₀alkyl, CO₂aryl (e.g.. CO₂phenyl), CO₂aralkyl (e.g. CO₂ benzyl)

The term “heteroatom” or “hetero” as used herein in its broadest sense refers to any atom other than a carbon atom which may be a member of a cyclic organic group. Particular examples of heteroatoms include nitrogen, oxygen, sulfur, phosphorous, boron, silicon, selenium and tellurium, more particularly nitrogen, oxygen and sulfur.

The invention will now be described with reference to the following examples. However, it is to be understood that the examples are provided by way of illustration of the invention and that they are in no way limiting to the scope of the invention.

### EXAMPLES

### Chemicals and Materials

Isobutyronitrile, ethanolamine, cadmium acetate, valeroyl chloride, 2-chloroethylamine-hydrochloride, triethylamine (TEA), methyl trifluoromethanesulfonate (MeOTf), sulforhodamine B acid chloride, tetrabutylammonium hydroxide (TBA-OH, 40 wt.% solution in water), phenylethylamine, hydrocinnamic acid, 2-phenylethanol, benzoic acid, benzene sulfonic acid, phenylphosphonic acid, phenylboronic acid, aniline, benzylalcohol, phenol, ethylenediamine, 4-(aminomethyl)pyridine, p-xylylenediamine. 1-(3-Dimethylaminopropyl)-3-ethylcarbodiimide hydrochloride (EDC.HCl), N-hydroxysuccinimide (NHS), Dowex50WX8-400 ion exchange resin, porcine gelatin type A (bloom 300, average molecular weight 50-100 kDa), sodium fluorescein, bovine serum albumin (BSA), peroxidase from horseradish (HRP), and Anti-Human IgG (Fc specific) antibody produced in goat were purchased from Sigma-Aldrich. Sodium hyaluronate (HANa, 15-30 kDa, 90-130 kDa, and 500-750 kDa) was purchased from Contipro (Czech Republic). Anhydrous dichloromethane (DCM), acetone, methanol, and ethanol were purchased from Merck. All these chemicals were used as received. Ephrin-A1 protein construct (MW = 120 kDa) was synthesised in-house using CSIRO protein synthesis facilities.

Acetonitrile (ACN) and dimethyl sulfoxide (DMSO) were purchased from Sigma Aldrich and dried over micro-waved activated molecular sieves (Sigma Aldrich, 4 Å). Spectra/Por® dialysis membranes were purchased from SpectrumLabs. 2-methyl-2-oxazoline was purchased from Sigma-Aldrich. 2-isopropyl-2-oxazoline and 2-n-butyl-2-oxazoline were synthesized and purified in lab according to previously reported protocols [Macromolecules 2006, 39, 3509]. All these 2-alkyl-2-oxazoline were distilled twice under reduced pressure prior to polymerisation reaction.

### General Procedures

### Nuclear Magnetic Resonance Spectroscopy (NMR) Characterisation

Nuclear Magnetic Resonance spectroscopy (¹H-NMR) were employed to confirm the expected chemical structure of the synthesised polymers. The NMR experiments were performed on the devices Bruker Avance III HD 400 MHz spectrometer (¹H, 400.1 MHz), Bruker Avance III 400 MHz spectrometer (¹H, 400.1 MHz), and Bruker Avance III HD 500 MHz spectrometer (¹H, 500.5 MHz). ¹H NMR spectra were recorded in deuterated chloroform (CDCl₃) or deuterated oxide (D₂O). Samples for NMR spectroscopy were prepared by dissolving the analyte in deuterated solvent, as specified, and placing the solution into a 5 mm NMR tube. The experiments were performed at either 12 ± 0.1° C. or 25 ± 0.1° C., depending on the polymer cloud point. The data were processed using Bruker TopSpin v3.6 software. The chemical shifts δ of the individual peaks of the proton spectra are given in parts per million (ppm) and are calibrated to the internal residual proton signal of the deuterated solvent.

### Calculation of Degree of Substitution

The degree of substitution was defined as

$$\text{DS} \text{=} \frac{\text{Total number of POxa chains}}{\text{Total number of termination sites}} \times 100 \% \qquad \text{(Equation 1)}$$

For HA-POxa conjugates, DS was calculated from ¹H NMR spectra using the following equation:

$${\text{DS}}_{\text{HA-POxa}} = \frac{\left({I}_{1} / 2 + {I}_{2}\right) / \text{DP}}{{I}_{3}} \times 100 \% \qquad \text{(Equation 2)}$$

Where I₁ is the integration of the peak at δ 0.9-1.2 ppm (protons in iso-propyl groups); I₂ is the integration of the peak at δ 0.8-0.9 ppm (protons in n-butyl groups); and I₃ is the integration of the peak at δ 1.8-2.0 ppm (protons in acetyl group of HA).

DP is the degree of polymerisation, calculated as

$$\text{DP} \text{=} \frac{{I}_{1} / 6 + {I}_{2} / 3}{{I}_{4} / 3} \qquad \text{(Equation 3)}$$

Where I₄ is the integration of the methyl (CH₃) end group in the polymer chain (δ 3.1 ppm).

For Gelatin-POxa conjugates, DS was calculated from ¹H NMR spectra using the following equation:

$${\text{DS}}_{\text{Gelatin-POxa}} = \frac{{I}_{1} / 6 / D {P}_{i s o}}{227.1} \times 100 \% \qquad \text{(Equation 4)}$$

Where I₁ is the integration of the peak at δ 0.9-1.2 ppm (protons in iso-propyl groups). The NMR integration of the gelatin conjugates was calibrated as 78.5 for the aromatic peak at δ 7.3 ppm, assuming that 227.1 mmol of termination sites were presented in 100 gm of gelatin (MW of 100 kDa) [Handbook of gelatin, 2012]. DPᵢₛₒ is the average number of 2-isopropyl-2-oxazoline monomer, calculated from the integration of the methyl (CH₃) end group in the polymer chain (δ 3.1 ppm) and I₁.

### Gel Permeation Chromatography (GPC) Characterisation

Polymer solutions of 1 mg mL⁻¹ were prepared in the eluent and filtered through 0.45 µm filters prior to injection. Number (Mₙ) and weight average (M_{w}) molar masses were evaluated using Shimadzu LC Solution software. GPC analyses of polyoxazoline samples were performed in SHIMADZU GPC (Waters Styragel HT5, HT4, HT3, HT2 + guard styragel column) with N,N′ -dimethyl- acetamide (DMAc with 0.03% w/v LiBr) as the eluent at the flow rate of 1 mL min⁻¹. The GPC columns were calibrated with low dispersity polystyrene standards (Polymer Laboratories) ranging from 575 to 3,242,000 g mol⁻¹, and molar masses are reported as low dispersity polystyrene equivalents. A 3rd-order polynomial was used to fit the log Mₚ vs. time calibration curve, which was near linear across the molar mass ranges.

For aqueous GPC analyses of HA, HA-POxa conjugates, gelatin, and gelatin-POxa conjugates, three Eprogen CATSEC columns 100, 300, and 1000 (5 micron; 250 × 4.6 mm) and Eprogen CATSEC guard column 100 (7 microns; 250 × 4.6 mm) were used, using anionic buffer (containing 0.2 M sodium nitrate and 0.01 M sodium phosphate) at 0.3 ml min⁻¹ as the eluent. A differential refractive index detector was used calibrated with linear poly(ethylene glycol) standards.

### Rheological Characterisation

The conjugates were dissolved in PBS 1× (0.1 M, pH 7.4) to make solution of desired concentration in a 2 ml glass vial. The samples were vortexed several times to ensure complete mixing. The conjugate solution was gradually heated up from 4° C. to 37° C. by using a water bath and a simple vial-inverting method was employed to determine the occurrence of sol-gel transition. The sol and gel phases were defined as flowing liquid and non-flowing gel, respectively, when the test vial was inverted. The gelation reversibility of the conjugate solution was confirmed by repeating the inverting-vial test several times.

Rheological characterisation of the conjugates was carried out using an Anton Paar MCR 301 rheometer. Measurements were performed with a parallel-plate geometry (diameter 25 mm) and silicon oil was used on the outer edge to prevent water evaporation. The temperature sweep experiments were performed by measuring storage modulus (G′) and loss modulus (G″) at a frequency of 1 rad s⁻¹ and 1% strain over a temperature range from 1° C. to 55° C. Temperature was increased at a rate of 1° C. min⁻¹, and samples were allowed to equilibrate at 1° C. for 5 minutes before the measurement started. Gelation temperature was defined as the temperature where G′ and G″ cross over (G′ = G″ or tan δ = 1). The sample is in the liquid phase when G′ < G″ (or tan δ > 1), while the sample is in the hydrogel phase when G′ > G″ (or tan δ < 1).

### Measurement of LCST

Turbidity measurements were carried out on a Cary 50 Bio UV-visible spectrophotometer (Varian Co., USA) for 2% w/v aqueous polymer solutions. The cloud point was determined as the temperature corresponding to a 50% decrease in optical transmittance at a visible wavelength (λ = 500 nm). The solution temperature was increased by a rate of 1° C. min⁻¹ followed by a 15-min period of constant temperature to ensure equilibration. The lowest critical solution temperature of the polymer was determined by the minimum value of the cloud point temperatures within a range of polymer concentration in an aqueous liquid.

### Example 1: Synthesis of HA-POxa Conjugates

HA-POxa conjugate was prepared according to the following reaction Scheme 2.

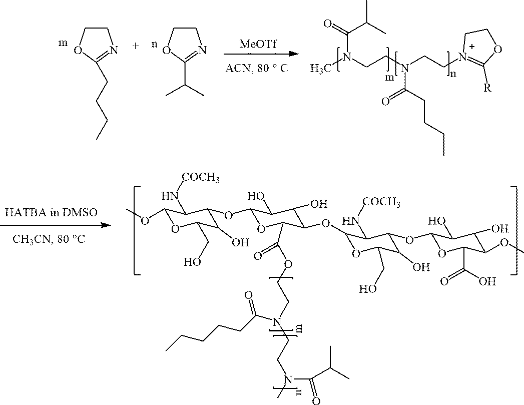

### Scheme 2: Synthesis of HA-POxa Conjugates

In a typical example, Dowex Resin 50WX8-100 (50 g) was washed several times and was further soaked overnight with DI water until the washing solution became clear. The resin was then filtered to remove water, and subsequently covered with 30 g of TBA-OH aqueous solution (40 wt.%). After one hour of shaking, the supernatant was removed and replaced with 30 g of fresh TBA-OH solution and placed overnight on an orbital shaker. Then, the resin was rinsed with large amount of DI water until the pH was below 10, followed by filtering off the water using a Gooch filter (number 2). Sodium hyaluronate (1 g) was dissolved in 100 mL of DI water and then filled in the resin-containing filter. The solution was left to flow through the resin and finally collected and lyophilized. The final product, tetrabutylammonium hyaluronate (HA-TBA), was kept in -20° C. until further use.

Next, 2-isopropyl-2-oxazoline (1 mL, or 8.84 mmol), 2-n-butyl-2-oxazoline (161 µL, or 1.25 mmol), MeOTf (13.8 µL, or 0.13 mmol), and acetonitrile (1.3 mL) were added in a microwave reaction vial (Biotage). The living cationic ring opening polymerisation was carried out in a microwave reactor at 140° C. for 30 minutes. For ‘one-pot’ conjugation method, calculated volume of living cationic polyoxazoline chain (3.6 mL, or 0.183 mmol) was added into a flask of HA-TBA dissolved in anhydrous DMSO (15 mL of 2% w/v solution). This step is performed under nitrogen gas using a glove box to minimise reaction with ambient moisture. The molar feed ratio of polyoxazoline chain to carboxylic group of HA-TBA for this experiment was calculated as 0.35. Then, the reaction mixture was stirred at 70° C. in oil bath for 24 hours. After termination, acetonitrile was removed by rotary evaporator and the remaining polymer solution was diluted in water for dialysis to remove DMSO. Dialysis was performed in DI water for 5 days with two changes daily using dialysis membranes with 50 kDa molecular weight cut-off. The final product was collected by freeze-drying and stored at -20° C. until further use.

Various HA-POxa conjugates were prepared in the same way, as seen in Table 1.

### Example 2: Characterisation of Rheological Properties of HA-POxa Conjugates At Different Concentrations

Solution of various concentrations (i.e. 1%, 3%, 7%, 12%, and 18% w/v) of HA-POxa conjugate was prepared by dissolving the dried conjugates synthesised according to Example 1 (Conjugate 6) in PBS 1x. The rheological properties including the storage modulus (G′) and loss modulus (G″) were characterised according to the Rheological Characterisation Procedure described previously.

As shown in FIG. 1, both moduli measured at 37° C. increased drastically when the solution concentration was increased. The tan δ measured at 37° C. was found to decrease with solution concentration (FIG. 2). Hydrogel formation, indicated by tan δ < 1, was observed at all the concentrations except 1% w/v.

The storage modulus was higher than 100 Pa for the solutions of 7% w/v and above.

### Example 3: Characterisation of Rheological Properties of HA-POxa Conjugates At Different Molecular Weights of HA and Varying Polymer LCST

Solutions of various concentrations (i.e. 7%, 12%, and 18% w/v) of HA-POxa conjugates with the same DS of 16% (Conjugate 2, Conjugate 7, and Conjugate 11 synthesised according to Example 1) was prepared by dissolving the dried conjugates in PBS 1x. The rheological properties including the storage modulus (G′) and loss modulus (G″) were characterised according to the Rheological Characterisation Procedure described previously.

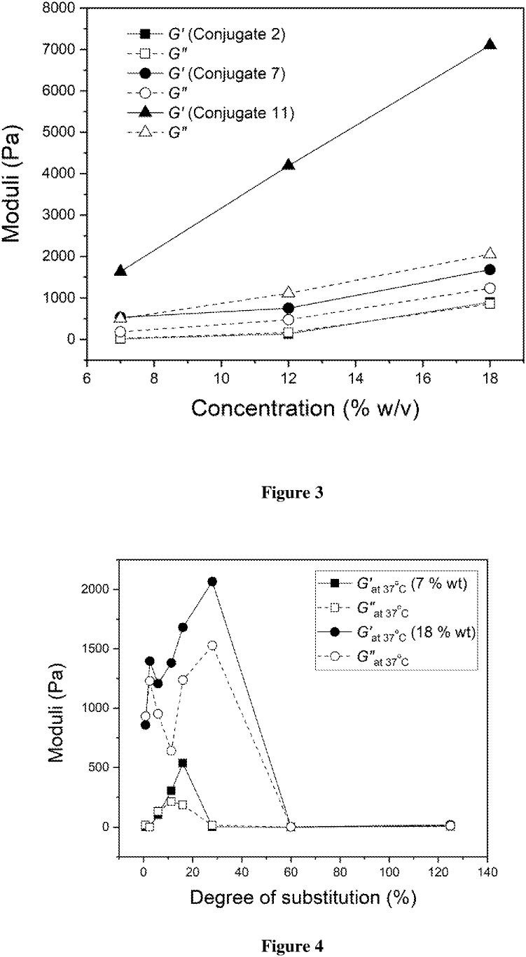

It was found that the molecular weight of HA and the LCST of POxa are important factors to determine the thermo-gelation properties of the HA-POxa conjugates, as shown in FIG. 3. The gelation ability as well as rheological moduli of the conjugates were improved by using HA molecules of longer molecular weight. As seen in FIG. 3, gelation at 37° C. of Conjugate 2 (100 kDa HA) was only observed at 18% w/v, and not at 7% and 12% w/v concentrations. In contrast, Conjugate 7 which was synthesised by using higher molecular weight of HA (500 kDa HA) was able to form into hydrogels at 37° C. at all the three concentrations. The storage and loss moduli of Conjugate 7 were significantly higher than those of the Conjugate 2 throughout the concentration range.

FIG. 3 also depicts that the LCST of POxa was tuneable based on the molecular ratio between 2-isopropyl-2-oxazoline and 2-n-propyl-oxazoline, which was used to change the gelation temperature of the HA-POxa conjugates. Conjugate 11 was synthesised by using POxa of lower LCST (LCST = 17° C.) exhibited higher G′ in comparison with Conjugate 7 made from POxa with LCST of 24° C.

### Example 4: Characterisation of Rheological Properties of HA-POxa Conjugates At Different Degree of Substitution (DS)

Solutions of different concentrations (i.e. 7% and 18% w/v) of HA-POxa conjugates with varying DS (Conjugate 3 - 10, synthesised according to Example 1) were prepared by dissolving the dried conjugates in PBS 1× at the appropriate concentration. The rheological properties including the storage modulus (G′) and loss modulus (G″) were characterised according to the Rheological Characterisation Procedure described previously.

FIG. 4 showed that the rheological properties of the hydrogel are strongly dependent on the degree of substitution (Conjugate 3 -10). The storage G′ and loss moduli G″ increased initially with increasing DS until unexpectedly reaching a maximum G′ value at DSₘₐₓ, and then decreased when DS was further increased from this point. Moreover, the DSₘₐₓ of the conjugates could be regulated by changing the solution concentration, whereby it shifted from DSₘₐₓ = 16% (Conjugate 7) to DSₘₐₓ = 28% (Conjugate 8) for the solution concentration of 7% w/v and 18% w/v, respectively. It is noteworthy that there is an optimal range of DS that imparts the formation of hydrogel (indicated by tan δ < 1) with the storage modulus above 100 Pa at 37° C. The optimal DS range of the 18% w/v conjugate solution (DS = 2.5% - 28%, Conjugates 4 - 8) is wider compared to that of the 7% w/v one (DS = 5.9% - 16%, Conjugates 5 - 7).

### Example 5: In-vitro Prolonged HRP Release

In a typical example, 3 mg of HRP was dissolved in 150 µL of PBS 1x (pH 7.4, 0.01 M) to prepare a 2% w/v HRP solution in a glass vial. Then, 27 mg of Conjugate 7 and Conjugate 11 were used to make an aqueous solution of 18% w/v (gently vortexed and left overnight at 4° C. to completely dissolve).

After the conjugates were completely dissolved, the aqueous liquid samples were placed in a 37° C. water bath for 5 minutes to form hydrogel, and subsequently 1.5 mL of 37° C. PBS 1x was slowly added in the vial. HRP release kinetics was monitored over two weeks. At each time point, 200 µL of the supernatant was replaced with 200 µL of fresh 37° C. PBS to keep the volume of the whole system constant. The collected 200 µL HRP solution was divided into 3 × 60 µL in 96-well plates, and the HRP concentration of each sample was measured using a plate reader (Bio-Rad, Hercules, CA, USA) by recording the absorbance at 405 nm. A calibration curve of HRP concentration was generated at each time interval. Samples in triplicate were analysed for each experiment.

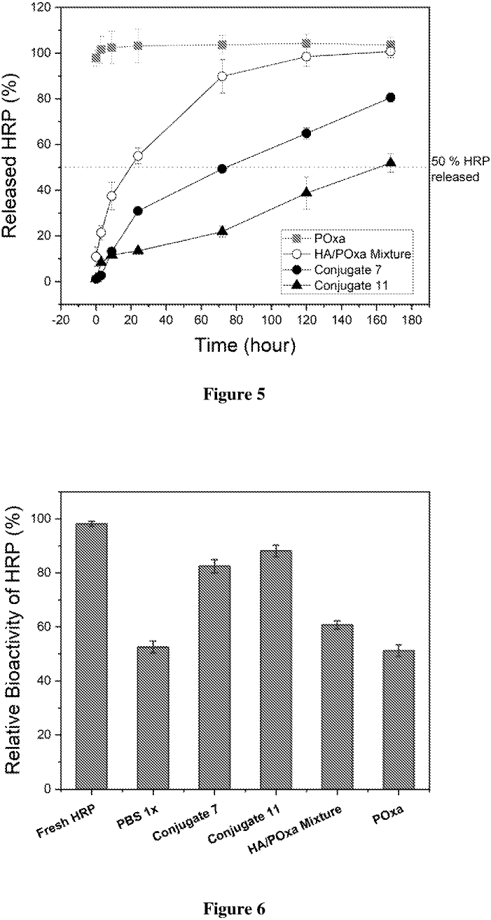

The HRP releasing kinetics of the conjugate and control samples are shown in FIG. 5. Conjugate 7 and Conjugate 11 in 18% w/v hydrogel showed linear HRP release kinetic profiles, which are typically desired for therapeutic delivery. It took approximately 7 days, and 14 days for 50% of HRP to be released from hydrogel containing 18% w/v Conjugate 7 and Conjugate 11, respectively.

### Example 6: Bioactivity of HRP Released From the HA-POxa Conjugates

In a typical example, the enzymatic activity of HRP released from the conjugates at day 7 as described in Example 5 was measured by using UV-vis spectroscopy. The collected HRP was added to a solution containing 30% of H₂O₂ (5% v/v) and ABTS (0.5 mg mL⁻¹) in 100 µL of phosphate solution (pH 5). The oxidised ABTS•+ product was indicated by the green color change of the solution and was quantified by measuring the absorbance at 405 nm. A standard curve was obtained by measuring different known HRP concentrations and HRP bioactivity was calculated against this standard curve. Fresh HRP solution was used as a standard sample to depict 100% bioactivity, whereby the HRP solution after 7-days incubation at 37° C. (PBS 1×) serves as a control sample.

FIG. 6 showed the biological activity of the released HRP enzyme from the hydrogel samples after incubation at 37° C. for 7 days. Over 7 days HRP in an aqueous solution (PBS 1×) lost its bioactivity to 53%, relative to a fresh HRP solution.. In contrast, when the HRP was encapsulated inside a hydrogel containing 18% w/v Conjugate 7 and Conjugate 11, significant improvements in retaining the bioactivity of HRP were demonstrated (82% and 88%, respectively).

### Example 7: Synthesis of Gelatin-POxa Conjugates

The ‘one-pot’ conjugation method was also employed successfully in the synthesis of gelatin-polyoxazoline (Gelatin-POxa) conjugates. The synthesis procedures are shown in Scheme 3.

[image]

### Scheme 3: Synthesis of Gelatin-POxa Conjugates

Typically, gelatin contains amino acid units that exhibit carboylic acid, thiol, and amine side-functionalities at different amounts. In order to decrease the zwitterionic interaction and increase the nucleophilicity of these functional groups, the carboxylic acid (Glu and Asp) of the gelatin side-groups were converted to primary amines. Porcine gelatin was simply chemically pre-modified with ethylenediamine to increase the number of amine groups, in order to provide aminated gelatin (chemically modified biomacromolecule with amino nucleophilic functional groups) for higher selectivity and efficiency in POxa ‘one-pot’ conjugation. In detailed synthesis procedures, 20 g of type A porcine gelatin was dissolved in 500 ml MES buffer solution (pH 6) at 50° C. After complete dissolution, the solution was cooled down to room temperature under vigorous stirring and ethylenediamine (62.26 ml) was then added dropwise while at the same time HCl (32%) was used to maintain the pH at 6. After that, EDC·HCl (10 g) was added in portions and the reaction was let to proceed for 24 hours at room temperature. Afterwards, the reaction flask was placed in a rotary evaporator at reduced pressure and 75° C. to reduce the reaction volume prior to dialysis. Dialysis was performed against DI water using 1 kDa MWCO for four days with three changes daily for the first two days and one change daily for the second two days. The pre-aminated gelatin was subsequently lyophilized and stored at 4° C. until further use.

In a typical example, 2-isopropyl-2-oxazoline (4 mL, 35.3 mmol), 2-n-butyl-2-oxazoline (1.4 mL, 11.1 mmol), MeOTf (15 µL, 0.14 mmol), and acetonitrile (6.2 mL) were added in a microwave reaction vial (Biotage). The living cationic ring opening polymerisation was carried out in a microwave reactor at 140° C. for 2.5 hours. For termination step, calculated volume of living cationic polyoxazoline chain (0.58 mL, 0.007 mmol) was added into a flask of pre-aminated gelatin in anhydrous DMSO (5 mL of 2% w/v solution). This step was performed under nitrogen gas using a glove box to minimise reaction with ambient moisture. The molar feed ratio of polyoxazoline chain to the total number of nucleophilic groups (227.1 mmol amino per 100 g of gelatin) of aminated gelatin for this experiment was calculated as 0.03. Then, the reaction mixture was stirred at 40° C. in oil bath for 24 hours. After ‘one-pot’ conjugation, acetonitrile was removed by rotary evaporator and the remaining polymer solution was diluted in water for dialysis to remove DMSO. Dialysis was performed in DI water for 5 days with two changes daily using dialysis membranes with 50 kDa molecular weight cut-off. The final gelatin-POxa conjugate product was collected by freeze-drying and stored at -20° C. until further use.

Various HA-POxa conjugates were prepared in the same way, as seen in Table 2.

### Example 8: Characterisation of Rheological Properties of Gelatin-POxa Conjugates At Different Degree of Substitution

Solution of 20% w/v concentration of Gelatin-POxa conjugates with varying DS (Conjugate 13 - 19, synthesised according to Example 7) was prepared by dissolving the dried conjugates in PBS 1x. The rheological properties including the storage modulus (G′) and loss modulus (G″) were characterised according to the Rheological Characterisation Procedure described previously.

The relationship between DS and rheological moduli of the GLT-POXA conjugates (20% w/v) were shown in FIG. 7. Similar finding was unexpectedly observed in the hydrogels containing HA-POxa conjugates, whereby there is an optimal range of DS for gelation of Gelatin-POxa conjugates to occur. The DSₘₐₓ value, where the rheological moduli of Gelatin-POxa conjugate reached its maximum, is recorded at DS = 11%. The detail properties of this sample series are listed in Table 2.

### Example 9: Preparation and Rheological Property of a Physical Mixture From HA-POxa and Pre-Aminated Gelatin

In a typical example, Mixture 1 was prepared by adding 35 mg of Conjugate 6 and 25 mg of aminated gelatin to 0.5 mL of PBS 1x. The mixture was gently vortexed and left overnight at 4° C. to completely dissolve. The final solution of Mixture 1 was consisted of 7% w/v of Conjugate 6 and 5% w/v pre-aminated gelatin, which was synthesized as described in Example 7. The rheological properties including the storage modulus (G′) and loss modulus (G″) were characterised according to the Rheological Characterisation Procedure described previously. FIG. 8 shows the rheological properties of these samples as a function of temperature. While Conjugate 6 (7% w/v) showed a gelation temperature at 35.8° C., Mixture 1 shifted the gelation temperature to 34.3° C. Nevertheless, the storage modulus (G′) of the Mixture 1 is unexpectedly lower than a single solution of 7% w/v of Conjugate 6, although the solid content of Mixture 1 was higher due to additional 5% w/v pre-aminated gelatin. Moreover, Mixture 1 exhibited two phase transitions, whereby the gel-sol transition occurring at 26.8° C. is predominated by gelatin and the sol-gel transition occurring at 34.3° C. predominantly due to the LCST of the HA-POxa conjugate. This is an exemplary evidence that the gelation properties of a conjugate (gelation temperature or G′) could be adjusted by a mixture of conjugates prepared by the method described in the invention as well.

TABLE 1

Synthesis of HA-POxa conjugates

| Conjugate | HA (kDa) | POxa | | | DS (%) | Concentration (w/v %) | Gelation temperature^{c} (°C) | G′ at 37° C. (Pa) | G″ at 37° C. (Pa) | Tan δ at 37° C. (G″/G′) | Suitable for hydrogel depot 1) tan δ_{at 37° C.} ≤ 1 2) G′_{at 37° C.} ≥ 100 Pa) |
|---|---|---|---|---|---|---|---|---|---|---|---|
| | | ―a M ₙ (kgmol⁻¹) | Composition^{b} ([M₁]/[M₂]) | LCST (°C) | | | | | | | |
| 1 | 20 | 13.6 | 100/14 | 25 | 18 | 7 | 39.5 | 10 | 28 | 2.80 | No |
| | | | | | | 12 | 39.7 | 55 | 166 | 3.01 | No |
| | | | | | | 18 | 38.1 | 637 | 884 | 1.39 | No |
| 2 | 100 | 13.6 | 100/14 | 25 | 16 | 7 | 39.3 | 15 | 25 | 1.68 | No |
| | | | | | | 12 | 38.3 | 136 | 169 | 1.25 | No |
| | | | | | | 18 | 36.7 | 899 | 862 | 0.96 | Yes |
| 3 | 500 | 13.6 | 100/14 | 25 | 0.8 | 7 | 52 | 34 | 16.5 | 7.1 | No |
| | | | | | | 18 | 38.2 | 858 | 932 | 1.09 | No |
| 4 | 500 | 13.6 | 100/14 | 25 | 2.5 | 7 | NO | 0.02 | 0.75 | 36 | No |
| | | | | | | 18 | 36 | 1396 | 1229 | 0.88 | Yes |
| 5 | 500 | 13.6 | 100/14 | 25 | 5.9 | 7 | 37.9 | 103 | 132 | 1.29 | No |
| | | | | | | 18 | 35 | 1207 | 952 | 0.79 | Yes |
| 6 | 500 | 13.6 | 100/14 | 25 | 11.3 | 1 | 40.3 | 0.05 | 0.6 | 12.8 | No |
| | | | | | | 3 | 36.3 | 7.5 | 5.5 | 0.73 | No |
| | | | | | | 7 | 35.7 | 304 | 211 | 0.7 | Yes |
| | | | | | | 12 | 34.8 | 503 | 256 | 0.51 | Yes |
| | | | | | | 18 | 34.5 | 1380 | 641 | 0.46 | Yes |
| 7 | 500 | 13.6 | 100/14 | 25 | 16 | 7 | 36.1 | 536 | 186 | 0.35 | Yes |
| | | | | | | 12 | 35.8 | 757 | 479 | 0.63 | Yes |
| | | | | | | 18 | 33.9 | 1680 | 1236 | 0.74 | Yes |
| 8 | 500 | 13.6 | 100/14 | 25 | 28 | 7 | 40 | 3 | 15.4 | 5.13 | No |
| | | | | | | 18 | 33.6 | 2067 | 1527 | 0.74 | Yes |
| 9 | 500 | 13.6 | 100/14 | 25 | 60 | 7 | NO | 0.02 | 0.75 | 36 | No |
| | | | | | | 18 | 38 | 2.5 | 2.9 | 1.14 | No |
| 10 | 500 | 13.6 | 100/14 | 25 | 125 | 7 | 34 | 9 | 6.1 | 0.68 | No |
| | | | | | | 18 | 34 | 18.6 | 12.2 | 0.65 | No |
| 11 | 500 | 15.4 | 100/34 | 17 | 16 | 7 | 29.9 | 1630 | 506 | 0.31 | Yes |
| | | | | | | 12 | 28.9 | 4192 | 1116 | 0.27 | Yes |
| | | | | | | 18 | 27.7 | 7104 | 2047 | 0.29 | Yes |
| 12 | 500 | 15.4 | 100/34 | 17 | 5 | 7 | 27 | 1007 | 302 | 0.30 | Yes |

^{a} Number average molecular weight from GPC.
^{b} [M₁]/[M₂]: molar concentration ratio of 2-isopropyl-2-oxazoline to 2-n-butyl-2-oxazoline.
^{C} Gelation temperature was determined by rheometer when G′ = G″ (or tan δ = 1).

TABLE 2

Synthesis of Gelatin-POxa conjugates

| Conjugate | POxa | | | DS (%) | Concentration (w/v %) | Gelation temperature^{c} (°C) | G′ at 37° C. (Pa) | G″ at 37° C. (Pa) | Tan δ at 37° C. (G″/G′) | Suitable for hydrogel depot 1) tan δ_{at 37°C} ≤ 1 2) G′_{at 37°C} ≥ 100 Pa) |
|---|---|---|---|---|---|---|---|---|---|---|
| | -a Mₙ (kg mol⁻¹) | Composition^{b} ([M₁])/[M₂]) | LCST (°C) | | | | | | | |
| 13 | 39.6 | 100/34 | 15.8 | 0.6 | 20 | NO | 2 | 3.2 | 1.6 | No |
| 14 | 39.6 | 100/34 | 15.8 | 0.7 | 20 | NO | 0.9 | 2.8 | 3.1 | No |
| 15 | 39.6 | 100/34 | 15.8 | 2.8 | 20 | NO | 14 | 19 | 1.3 | No |
| 16 | 39.6 | 100/34 | 15.8 | 4 | 20 | 29 | 55 | 32 | 0.59 | No |
| 17 | 39.6 | 100/34 | 15.8 | 11 | 20 | 28 | 1048 | 474 | 0.45 | Yes |
| 18 | 39.6 | 100/34 | 15.8 | 16.4 | 20 | 30 | 150 | 97 | 0.65 | Yes |
| 19 | 39.6 | 100/34 | 15.8 | 25.5 | 20 | 26 | 468 | 260 | 0.56 | Yes |

^{a} Number average molecular weight from GPC.
^{b} [M₁]/[M₂]: molar concentration ratio of 2-isopropyl-2-oxazoline to 2-n-butyl-2-oxazoline.
^{c} Gelation temperature was determined by rheometer when G′ = G″ (or tan δ = 1).

### Comparative Example C1: Comparison of the Conjugation Selectivity by Using Different Nucleophilic Groups

The aim of this experiment was to compare different functional groups, in order to assess their nucleophilicity for ‘one-pot’ conjugation method described in the invention. Small molecules were used instead of larger MW biomacromolecule to simplify the quantification of the ‘one-pot’ conjugation efficiency using ¹H-NMR. Poly(2-methyl-2-oxazoline) was prepared by living cationic ring-opening polymerization (LCROP) method under nitrogen atmosphere according to the following reaction scheme, followed by its ‘one-pot’ conjugation using various small molecules to represent different functional groups.

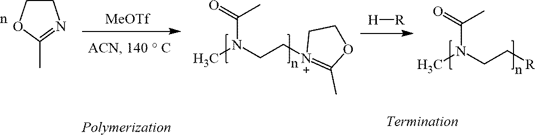

In a typical example, 2-methyl-2-oxazoline (5 mL, or 58.8 mmol), MeOTf (215 µL, or 1.96 mmol), and acetonitrile (9.5 mL) were added to a microwave reaction vial (Biotage). The polymer propagation was carried out in a microwave reactor at 140° C. for 60 minutes. Termination of LCROP was instantaneously carried out by conjugation of the living cationic with nucleophilic functional groups, which was demonstrated by taking 2 mL of the polymer solution to react with excessive amount of benzoic acid (2.67 mmol) in acetonitrile. The termination reaction was continued overnight at 70° C., and after that the polymer solution was diluted in 70 mL of water. Dialysis was performed in DI water for 8 weeks with two changes daily using dialysis membranes with 0.5 kDa molecular weight cut-off. The final product was collected by freeze-drying and stored at -20° C. until further use.

Various small-molecule conjugated poly(2-methyl-2-oxazoline) compositions were prepared in the same way, as seen in Table 3. The amino group seemed to exhibit the highest nucleophilicity for ‘one-pot’ conjugation with the living cationic POxa. Carboxylate, sulfonate, and phosphonate were shown to be sufficiently nucleophilic for ‘one-pot’ conjugation, which could be further increased if counterion salt was used to deprotonate the nucleophilic functional groups. In contrast, boronate and alcohol did not exhibit ‘one-pot’ conjugation with POxa. This example demonstrated that chemoselectivity could be achieved in biomacromolecule through the degree of nucleophilicity of the functional groups, i.e. carboxylate versus alcohol in hyaluronic acid.

TABLE 3

Small-molecule conjugated Poly(2-Methyl-2-Oxazoline)

| Small molecule | Nucleophilic functional group (NFG) | Conjugated polymer | Conjugation efficiency (%)^{#} |
|---|---|---|---|
| 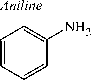 | Amino | 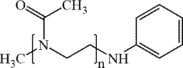 | 48 |
| 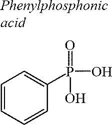 | Phosphonate | 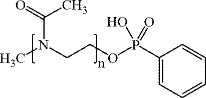 | 31 |
| 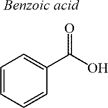 | Carboxylate | 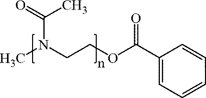 | 27 |
| 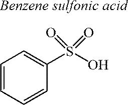 | Sulfonate | 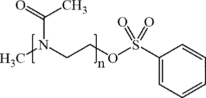 | 25 |
| 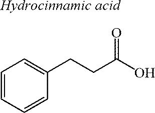 | Carboxylate | 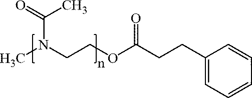 | 21 |
| 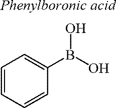 | Boronate | 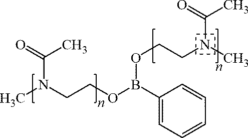 | 0 |
| 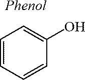 | Alcohol | 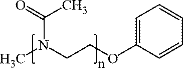 | 0 |
| 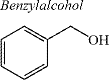 | Alcohol | 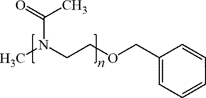 | 0 |

^{#}Conjugation efficiency determined by ¹H NMR end group analysis.

### Comparative Example C2: Characterization of Rheological Properties of a Physical Mixture of HA and POxa (HA/POxa Mixture)

For comparison, a physical mixture of 6.4 mg of HA (500 kDa) and 28.6 mg of a POxa (13.6 kg mol⁻¹; LCST = 24° C.) were added to make an aqueous solution of 7% w/v (HA/POxa Mixture). The solution was gently vortexed and left overnight at 4° C. to completely dissolve. The rheological properties including the storage modulus (G′) and loss modulus (G″) were characterised according to the Rheological Characterisation Procedure described previously.

The amount of HA and POxa of the HA/POxa Mixture was adjusted to match the molecular composition of HA and POxa in Conjugate 7. In this case, 35 mg of Conjugate 7 was dissolved in 0.5 mL of PBS 1× (pH 7.4, 0.01 M) to prepare a 7% w/v solution in a glass vial.

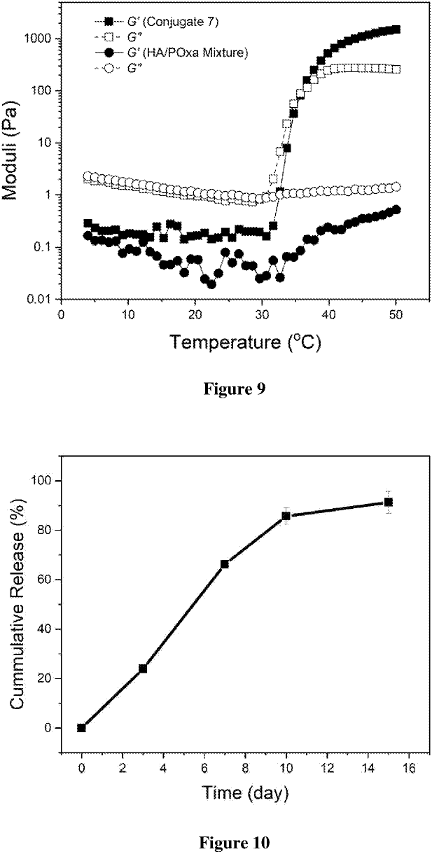

FIG. 9 shows the rheological properties of these samples as a function of temperature. While Conjugate 7 (7% w/v) showed a gelation temperature at 36° C., hydrogel formation was not observed for HA/POxa Mixture (7% w/v) over the entire range of temperature. This proved that HA-POxa conjugation is vital for the thermoreversible hydrogel formation process.

### Comparative Example C3: In-Vitro Prolonged HRP Release From HA/POxa Mixture and its Bioactivity Assessment

In a typical example, 3 mg of HRP was dissolved in 150 µL of PBS 1× (pH 7.4, 0.01 M) to prepare a 2% w/v HRP solution in a glass vial. Then, 27 mg of a POxa polymer (13.6 kg mol⁻¹; LCST = 24° C.) and a mixture of 4.9 mg of HA (500 kDa) and 22.1 mg of a POxa polymer (13.6 kg mol⁻¹; LCST = 24° C.) were added to the 2% w/v HRP solution to make the control samples, denoted as POxa and HA/POxa Mixture, respectively.

As shown in FIG. 5, a burst release behaviour was observed for sample POxa as 97% of HRP was released immediately after the addition of PBS. Although the POxa seemed to help the HA/POxa Mixture to precipitate in PBS at 37° C., burst release was still observed in the HRP release kinetic profile (50% release after 1 day). Apparently, since a hydrogel was not formed by POxa and HA/POxa Mixture, the HRP payload could not be sufficiently protected by these two polymers alone (FIG. 6). About less than 60% bioactivity was measured after the protein was incubated with the POxa and HA/POxa Mixture at 37° C. for 7 consecutive days, which was at the similar level as an aqueous solution of the HRP (PBS 1x).

### Example 10: In-vitro Prolonged Release of BSA (the Bioactive Agent)

In a typical example, 5 mg of Conjugate 12 was dissolved in 50 µL of BSA solution (400 µg/mL in PBS 1×) to make a BSA-loaded hydrogel of 10% w/v conjugate. After the conjugate were completely dissolved, the aqueous liquid samples were placed in a 37° C. water bath for 5 minutes to form a hydrogel, and subsequently 1 mL of 37° C. PBS 1x was slowly added in the vial. BSA release kinetics was monitored over two weeks. At each time point, 400 µL of the supernatant was replaced with 400 µL of fresh 37° C. PBS to keep the volume of the whole system constant. The protein concentration of the collected samples was measured using a Micro BCA™ Protein Assay Kit (Thermo Fisher Scientific). Samples in triplicate were performed for the experiment.

The BSA release kinetics of the conjugate is shown in FIG. 10. Conjugate 12 (10% w/v hydrogel) showed a near linear BSA release kinetic profile up to 10 days.

### Example 11: In-Vitro Prolonged Release of Ephrin-A1 Protein Construct (MW = 120 kDa)

In a typical example, ephrin-A1 construct (10 µg/mL) and anti-human IgG (Fc specific) antibody (20 µg/mL) were fused together at 37 oC for 2 hours. 100 µL of this solution was then used to dissolve 7 mg of Conjugate 12 to make an Ephrin-Al-loaded hydrogel sample of 7% wt/v conjugate. After the conjugate were completely dissolved, the aqueous liquid samples were placed in a 37° C. water bath for 5 minutes to form a hydrogel, and subsequently 400 µL of 37° C. PBS 1× was slowly added in the vial. Protein release kinetics was monitored over three weeks. At each time point, 200 µL of the supernatant was replaced with 200 µL of fresh 37° C. PBS to keep the volume of the whole system constant. The protein concentration of the collected samples was measured using a Micro BCA™ Protein Assay Kit (Thermo Fisher Scientific). Samples in triplicate were performed for the experiment.

The ephrin-A1 release kinetics of the conjugate is shown in FIG. 11. Conjugate 12 (7% w/v hydrogel) showed a near linear BSA release kinetic profile up to 14 days.

### Example 12: In-Vitro Prolonged Release of Sodium Fluorescein (Small Molecule)

In a typical example, 100 µg of sodium fluorescein and 18 mg of Conjugate 6 were dissolved in 100 µL of PBS 1× to make a solution of 18% w/v conjugate. After the conjugate were completely dissolved, the aqueous liquid samples were placed in a 37° C. water bath for 5 minutes to form a hydrogel, and subsequently 900 µL of 37° C. PBS 1× was slowly added in the vial. Fluorescein release kinetics was monitored over six weeks. At each time point, 300 µL of the supernatant was replaced with 300 µL of fresh 37° C. PBS to keep the volume of the whole system constant. The concentration of fluorescein was quantified by measuring the absorption spectra of the collected samples at 480 nm. Samples in triplicate were performed for the experiment.

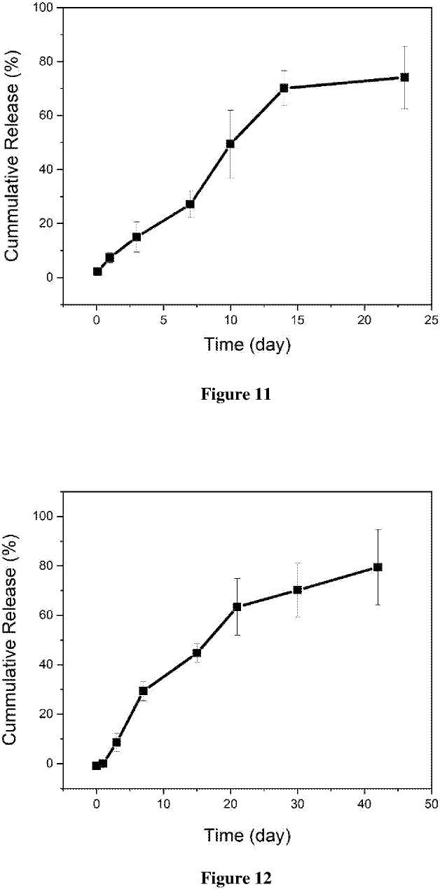

The release kinetics of fluorescein from the hydrogel conjugate is shown in FIG. 12. Conjugate 6 (18% w/v hydrogel) showed a sustained release kinetic profile with 80% of the encapsulated Fluorescein being released over 6 weeks.

### Comparative Example C4: Synthesis of Amine-Terminated POxa by ‘One-Pot’ Conjugation of Living Cationic Ring-Opening Polymerization Using Various Diamine Molecules

In principle, biomacromolecule-POxa conjugates could be synthesized by several indirect-conjugation methods employing two or more reaction steps. For example, it would be possible to synthesize HA-POxa conjugates by first synthesizing amine-end-functionalized POxa using direct termination of living cationic ring-opening polymerization with excess amount of diamine molecules, followed by the conjugation of the amine-terminated POxa to the carboxylic groups of HA molecule via 1-ethyl-3-(3-dimethylaminopropyl)carbodiimide (EDC) / N-Hydroxysuccinimide (NHS) chemistry as shown in reaction scheme 4.

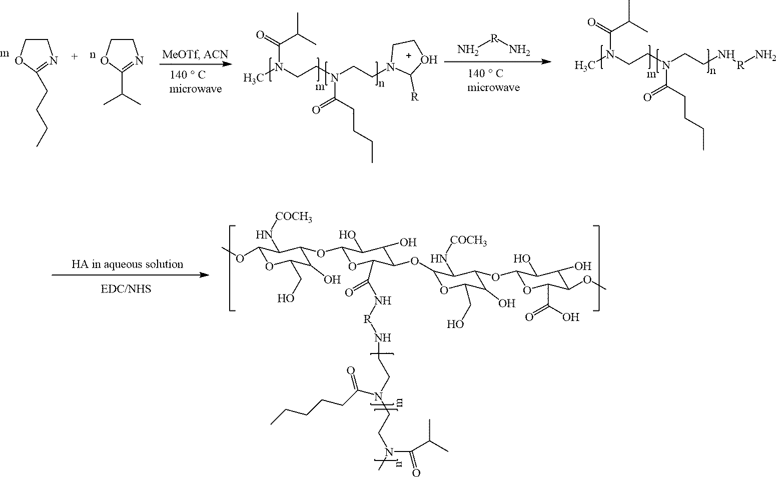

### Scheme 4: Synthesis of HA-POxa Conjugate by Using Two-Step Conjugation Method

An attempt to synthesize HA-POxa conjugate, using the two-step conjugation scheme mentioned above, was undertaken in order to compare the difference between this indirect method to the ‘one-pot’ one of the current invention. The purpose of this experiment was to evaluate the termination efficiency of different diamine molecules of varying nucleophilicities with poly(2-isopropyl-2-oxazoline)-co-poly(2-n-butyl-2-oxazoline) thermoresponsive copolymer.

In a typical example, 2-isopropyl-2-oxazoline (1 mL, or 8.84 mmol), 2-n-butyl-2-oxazoline (161 µL, or 1.25 mmol), MeOTf (13.8 µL, or 0.13 mmol), and acetonitrile (1.3 mL) were added in a microwave reaction vial (Biotage). The living cationic ring opening polymerisation was carried out in a microwave reactor at 140° C. for 30 minutes. For the direct termination step, calculated volume of ethylenediamine in acetonitrile (0.3 M) was added into the solution of living cationic polyoxazoline chain at a molar feed ratio of 5:1 (excess ethylenediamine). All the preparation steps were performed under nitrogen gas using a glove box to minimise reaction with ambient moisture. The termination was carried out in a microwave reactor at 140° C. for 2 hours. After that, the polymer solution was diluted in water and undergone dialysis. Dialysis was performed in DI water for 8 weeks with two changes daily using dialysis membranes with 1 kDa molecular weight cut-off. The final product was collected by freeze-drying and stored at -20° C. until further use.

Termination with other diamine molecules including p-xylylenediamine and 4-(aminomethyl)pyridine were also attempted using the same reaction, as shown in Table 4. However, the conjugation was not formed between poly(2-isopropyl-2-oxazoline)-co-poly(2-n-butyl-2-oxazoline) thermoresponsive copolymer and diamine molecules.

TABLE 4

Synthesis of amine-terminated POxa by ‘one-pot’ conjugation method

| Small molecule | Polymer | Termination conditions | Molar feed ratio | Conjugation efficiency -DS (%)^{a} | Quantification of free amine group (µg/mg polymer)^{b} | -a Mₙ of conjugate (kg mol⁻¹)^{c} | PDI ^{c} |
|---|---|---|---|---|---|---|---|
| Ethylene diamine 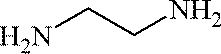 | P(iso/butyl) Oxa ^{#} | 140° C./ microwave 2 h | 5:1 | N/A ^{###} | -0.25 ± 0.47 | 12.9 | 1.16 |
| p-Xylylene diamine 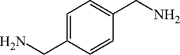 | Piso/butylO xa | 140° C./ microwave 2 h | 5:1 | 0 | N/A | 14.5 | 1.15 |
| 4-(Amino methyl)p yridine 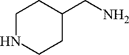 | Piso/butylO xa | 140° C./ microwave 2 h | 5:1 | 0 | N/A | 13.7 | 1.13 |

^{a}calculated from ¹H NMR spectra.
^{b} calculated from TNBSA assay (2,4,6-trinitrobenzene sulfonic acid, ThermoFisher Scientific).
^{c} calculated from GPC characterization.
^{#} P(iso/butyl)Oxa: poly(2-isopropyl-2-oxazoline)-co-poly(2-n-butyl-2-oxazoline) thermoresponsive copolymer.
^{###} not calculatable from ¹H NMR spectra because of peak overlaps.

### Comparative Example C5: Conjugation of Amine-End Functionalized Poly(2-Methyl-2-Oxazoline) With HA Via EDC/NHS Coupling

Here we aim to assess the efficiency of conjugating HA to POxa via a two-step route as outlined in the third reaction in Scheme 4.

In a typical experiment, 50 mg of HA (500 kDa; 0.125 mmol of carboxylic group) was dissolved in 10 mL of MES buffer (0.1 M; pH 6). After that, 386.5 mg of EDC.HCl (2.0 mmol) and 57 mg of NHS (0.5 mmol) were added and the solution was stirred vigorously for 4 hours at room temperature. Then, 230 mg of ethylene diamine-end functionalized poly(2-methyl-2-oxazoline) (28% DS; 18.5 kDa; 0.012 mmol) in 10 mL of MES buffer was added. The reaction was carried for 24 hours at room temperature. To collect the conjugate, dialysis was performed in DI water for 3 days with three water changes daily using dialysis membranes of 50 kDa molecular weight cut-off. The final product was collected by freeze-drying and stored at -20° C. until further use.

No evidence of success bioconjugation was observable in the ¹H nmr spectra of the reaction mixture after dialysis.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

1. A method of preparing a thermoresponsive polymer biomacromolecule conjugate, the method comprising:
preparing by living cationic ring opening polymerisation a thermoresponsive polymer selected from polyoxazoline, polyoxazine, or copolymer thereof, the so-formed thermoresponsive polymer having a living cation; and
reacting the living cation with a nucleophilic functional group selected from carboxylate, amino, sulfate, sulfonate, phosphate, phosphonate and thiol of a biomacromolecule to conjugate the thermoresponsive polymer to the biomacromolecule;
wherein in an aqueous liquid the so-formed thermoresponsive polymer biomacromolecule conjugate exhibits a gelation temperature and forms a hydrogel in that liquid above that gelation temperature.

2. The method according to claim 1, wherein the thermoresponsive polymer selected from poly(2-alkyl-2-oxazoline), poly(2-alkyl-2-oxazine) and copolymers thereof.

3. The method according to claim 1, wherein the thermoresponsive polymer selected from poly(2-ethyl-2-oxazoline), poly(2-isopropyl-2-oxazoline), poly(2-n-propyl-2-oxazoline), poly(2-n-butyl-2-oxazoline), poly(2-N,N-diethylamino-2-oxazoline), poly(n-propyl-2-oxazine) and copolymers thereof.

4. The method according to claim 1, wherein the thermoresponsive polymer prepared by the living cationic ring opening polymerisation has a molecular weight of between about 5 kDa and about 100 kDa.

5. The method according to claim 1, wherein the living cation reacts with an amino functional group of the biomacromolecule to conjugate the thermoresponsive polymer to the biomacromolecule.

6. The method according to claim 1, wherein the biomacromolecule is a polysaccharide or a polypeptide.

7. The method according to claim 1, wherein the living cation reacts with the nucleophilic functional group of the biomacromolecule to conjugate the thermoresponsive polymer to the biomacromolecule at a degree of substitution ranging between about 5% and about 30%.

8. A method of forming a hydrogel, the method comprising:
providing an aqueous liquid solution of thermoresponsive polymer biomacromolecule conjugate prepared in accordance with claim 1 the thermoresponsive polymer biomacromolecule conjugate exhibiting in the aqueous liquid a gelation temperature; and
raising the temperature of the aqueous liquid comprising the thermoresponsive polymer biomacromolecule conjugate to above the gelation temperature so as to promote formation of the hydrogel.

9. The method according to claim 8, wherein the gelation temperature ranges from about 20° C. to about 38° C.

10. The method according to claim 8, wherein the thermoresponsive polymer biomacromolecule conjugate is provided in the aqueous liquid at a concentration ranging between about 5% v/w to about 40% v/w.

11. The method according to claim 8, wherein the hydrogel in the aqueous liquid has a storage modulus (G′) of greater than 100 Pa as measured by a rheometer at a temperature at or above the gelation temperature.

12. The method according to claim 8, wherein the so formed hydrogel has a storage modulus (G′) of greater than 100 Pa at 37° C. as measured by a rheometer.

13. The method according to claim 8, wherein the aqueous liquid further comprises a biologically active agent and formation of the hydrogel encapsulates the agent within the hydrogel.

14. The method according to claim 13, wherein the biologically active agent is selected from antibiotics, antimicrobial agents, anti-viral agents, anaesthetics, steroidal agents, anti-inflammatory agents, anti-neoplastic agents, antigens, vaccines, antibodies, growth factors, decongestants, antihypertensives, sedatives, birth control agents, progestational agents, anticholinergics, analgesics, anti-depressants, anti-psychotics, β-adrenergic blocking agents, diuretics, cardiovascular active agents, vasoactive agents, non-steroidal anti-inflammatory agents, nutritional agents and prostaglandin.

15. The method according to claim 8, wherein the aqueous liquid further comprises microparticles and/or nanoparticles and formation of the hydrogel encapsulates the microparticles and/or nanoparticles within the hydrogel.
